(19) United States
(12) Patent Application Publication
(10) Pub. No.: US 2017/0209489 A1
(43) Pub. Date: Jul. 27, 2017

(54) FAST ACTING INHIBITOR OF GASTRIC ACID SECRETION WITH ENHANCED ACTIVITY

(71) Applicant: YALE UNIVERSITY, NEW HAVEN, CT (US)

(72) Inventor: John P. Geibel, Branford, CT (US)

(21) Appl. No.: 15/128,049

(22) PCT Filed: Mar. 31, 2015
(86) PCT No.: PCT/US2015/023499

§ 371 Date: Sep. 21, 2016

Related U.S. Application Data
(60) Provisional application No. 61/972,658, filed on Mar. 31, 2014.

(51) Int. Cl. A61K 33/30; A61K 45/06

(52) U.S. Cl. CPC A61K 33/30; A61K 45/06

(57) ABSTRACT

The present invention relates to the discovery that a hydrated salt form of zinc exhibits enhanced activity in reducing the acidity of gastric juice in a subject in need when compared to typical zinc salts. According to the invention, a zinc salt hydrate may be used alone or in combination with an additional zinc salt hydrate. Optionally these zinc salt hydrate(s) may be combined with at least one additional zinc salt. The zinc salt hydrate may be further combined with one or more of a protein pump inhibitor (PPI), H2 blocker, anti-H. pylori antibiotic/antimicrobial, cytoprotective agent or a combination agent or additional therapeutic agents for providing fast action with optional long duration effect in reducing gastric acid secretion. In addition, the proposed methods are useful for treating patients who are non-responsive to proton pump inhibitors (PPI) and as an alternative to traditional therapies or conditions which are caused by rapid and complete inhibition of secretagogue induced acid secretion.

### RELATED APPLICATIONS

The present application claims priority from provisional application U.S. 61/972,658, filed Mar. 31, 2014, of identical title, the entire contents of which are incorporated herein.

### FIELD OF THE INVENTION

The present invention relates to the surprising discovery that a hydrated salt form of zinc, for example, Zinc chloride monohydrate, Zinc chloride dihydrate, Zinc chloride trihydrate, Zinc Chloride quatrahydrate, Zinc acetate dihydrate, Zinc citrate dihydrate, zinc citrate trihydrate, zinc gluconate monohydrate, zinc gluconate dihydrate, zinc gluconate trihydrate, Zinc bromide dihydrate, Zinc fluoride dihydrate, Zinc nitrate hydrate, Zinc perchlorate hexahydrate, Zinc tetrafluoroborate hydrate, Zinc p-toluenesulfate hydrate, zinc sulfate monohydrate and in particular zinc sulfate heptahydrate (ZnSO₄.7H₂O), exhibits unexpectedly enhanced activity at very low dosages (e.g. in some cases at levels 1/100 or even 1/1000 a typical zinc salt) in reducing the acidity of gastric juice in a patient or subject in need when compared to typical non-hydrated zinc salts, including zinc chloride, zinc acetate, zinc gluconate and zinc sulfate, among others. In the present invention, at least one zinc salt hydrate, often zinc sulfate heptahydrate, may be used alone or in combination with another zinc salt hydrate, optionally in combination with at least one additional zinc salt non-hydrate, including a water soluble zinc salt non-hydrate. A zinc salt hydrate, often zinc sulfate heptahydrate, is used alone, in a combination of zinc salt hydrates or optionally, with an additional zinc salt(s), may be further combined with one or more of a protein pump inhibitor (PPI), antacid, H2 blocker, anti-H. pylori antibiotic/antimicrobial, cytoprotective agent or a combination agent, acetyl salicylic acid, a blood thinning agent or mixtures thereof as otherwise described herein for providing fast action with optional long duration effect in reducing gastric acid secretion, including acid secretion in the fundus (by inhibiting vacuolar H⁺-ATPase or H⁺/K⁺-ATPase) and upper body region of the stomach (by inhibiting H⁺/K⁺-ATPase), thus raising the pH of the stomach during resting phase as well as decreasing the duration of stomach acid release during a secretagogue phase and for treating conditions including gastroesophogeal reflux disease (GERD), non-erosive reflux disease (NERD), Zollinger-Ellison syndrome (ZE disease), ulcer disease, and gastric cancer, as well as preventing or reducing the likelihood of ulcer disease. In addition, the present methods are useful for treating patients who are non-responsive to proton pump inhibitors (PPI) and as an alternative to traditional therapies or conditions which are caused by rapid and complete inhibition of secretagogue induced acid secretion. In addition, the present compounds may be used in any individual who shows compromise in copper levels (low copper) or who wishes to limit copper intake from another source. In certain embodiments, a copper salt is supplemented in the patient taking the zinc salt, but generally at a time at least one hour after and preferably several hours (at least 1, 2, 3, 4, 5, 6, 7, 8 or more hours) after the zinc salt is administered. Pursuant to the present invention, it has unexpectedly been discovered that composition which comprises a zinc salt hydrate, often zinc sulfate heptahydrate, is unexpectedly more active on a mole to mole basis than compositions which comprise a number of other zinc salts (non-hydrated forms) which have been identified and used in this context and more active on a mole to mole basis with its own zinc salt non-hydrate. In particular, zinc sulfate heptahydrate exhibits unexpectedly exceptional activity at concentrations far lower than other zinc salts, including zinc chloride, zinc aceate, zinc gluconate and zinc sulfate monohydrate, among others.

The present invention also relates to the use of at least one zinc salt hydrate, alone or in combination, preferably, zinc sulfate heptahydate, alone or in combination with another zinc salt hydrate and/or at least one additional water soluble zinc salts (non-hydrates), administered in combination with a therapeutic compound or agent (second therapeutic agent) which may be delivered orally with enhanced bioavailability (compared to compounds which are administered in the absence of a zinc salt) or other favorable benefits. In addition, therapeutic agents which exhibit sensitivity to low pH may be advantageously orally administered in combination with an effective amount of at least one zinc salt hydrate, preferably zinc heptahydrate alone or in combination with at least one additional zinc salt hydrate and/or water soluble zinc salt. Compositions according to the present invention exhibit greater bioavailability of the active agent when formulated in combination with at least one zinc salt hydrate, preferably zinc sulfate heptahydrate alone or in combination with another zinc salt hydrate and/or a water soluble zinc salt in oral dosage form than when administered in the absence of a zinc salt.

### BACKGROUND OF THE INVENTION

The generation of concentrated 0.16N hydrochloric acid by the mammalian parietal cell involves a complex combination of neuronal and hormonal regulatory feedback loops¹⁻³. Following activation of the cell there is a complex cellular transfer of ions that allows for the formation of acid⁴⁻⁷. A disruption in any of these components (secretory receptors, or ion transporters) can lead to either a cessation in the secretion of acid, or in the hypersecretion of acid. In the latter over 30 million patients per year suffer from symptoms of acid related diseases with the numbers increasing yearly⁸⁻¹¹. Clinically the uncontrolled release or the continued hypersecretion of acid can lead to changes in both gastric and intestinal epithelium, but can in more serious cases lead to erosions of the esophagus that can result in metaplasia and death¹²⁻¹⁴. Recent evidence has also emerged that prolonged recurrent periods of hypersecretory states can lead to gastric carcinoid formation¹⁵.

In an attempt to design therapies to prevent hyperacid secretion a variety of approaches have been employed in recent years with two of the most successful being: a) inhibition of the Histamine receptor on the basolateral membrane of the parietal cell, b) proton pump specific drugs targeted against the H⁺,K⁺-ATPase (the so called proton pump inhibitors; PPI)¹⁶⁻¹⁸ Both of these therapies have greatly improved the quality of life for patients suffering from this disease, however there is an ever increasing number of patients that have experienced recurrent disease while still taking the drugs^{19, 20}. Despite their high degree of efficacy and worldwide clinical use, failure in the treatment of acid related diseases has been reported and the degree and speed of onset of symptom relief are important to patients²¹. It has been estimated that about 30% of GERD patients remain symptomatic on standard dose of PPI²². Furthermore PPI's have a short plasma half life which often leads to nocturnal acid breakthrough²³. Therapeutic oral doses of PPIs reach steady state and thus achieve their maximal effective levels after 4-5 days with typical dosing regimens²⁴. This slow and cumulative onset of effect of PPIs relates to their ability to inhibit only those pumps which are active when the PPI drug is available. After PPI administration, there is a return of acid secretion that is partly due to de novo synthesis of the enzyme²⁵.

Zinc is an essential part of the diet that all cells require in order to maintain membrane integrity and function. Deficiency in intracellular zinc leads to apoptotic events, and cell death²⁶⁻³⁰. Previous studies have investigated the potential role of zinc in the proliferation and generation of the protective barrier, namely the mucous gel layer at the surface of the stomach³¹⁻³⁴. These studies falsely attributed the reduction in acid secretion to an increase in the thickness of the gel layer.³³⁻³⁵. Continued administration of a zinc salt may require an increase in copper intake in order to address copper deficiency.

Gastric acid aids protein digestion; facilitates the absorption of iron, calcium, and vitamin B12; and prevents bacterial overgrowth. When levels of acid and proteolytic enzymes overwhelm the mucosal defense mechanisms, ulcers occur. To avoid damage that is associated with these harsh conditions, gastric acid must be finely regulated by overlapping neural (e.g. acetylcholine), hormonal (e.g. gastrin and ghrelin), and paracrine (e.g. histamine and somatostatin) pathways, and more recently via the Calcium Sensing Receptor. Any long term alterations in any of these regulatory pathways leads to cell and tissue destruction and clinical manifestations such as peptic ulcer diseases, or gastroesophageal reflux disease (GERD). Two methods are commonly employed to treat the overproduction of acid: a) surgically, by elimination of the neuronal element (vagotomy) or b) pharmacologically, either through histamine 2 receptor antagonists or proton pump inhibitors (PPI's) or a combination of both.

PPI's such as omeprazole are irreversible inhibitors of the gastric H⁺,K⁺-ATPase, recently various derivatives of the parent compound omeprazole that bind to multiple cysteine residues on the exofacial surface of the H⁺,K⁺-ATPase have been developed in hopes of having a tighter molecular binding, and longer action have been employed. Both rabeprazole, and lansoprazole are examples of these multiple binding drugs and are activated in the acidic lumen of the gastric gland and modify the cysteine residues located on the luminal surface of the H⁺,K⁺-ATPase. In the resting cell the acid secreting pumps are internalized in a system of tubular vesicles, and are in such a conformational state that the PPIs can only inhibit the H⁺,K⁺-ATPases which have already been activated and transferred to the apical surface of the parietal cell.

Although optimizing pharmacological profiles within the PPI class may provide some clinical benefit, other areas of research may prove to be more fruitful and furthermore the fine tuning of the acid secretory process is still not completely understood and remains an important target for therapies to modulate gastric acid secretion.

Zinc is required for a large number of biological processes including gene expression, replication, membrane stability, hormonal storage and release and as a catalytic component for enzymes. There has been no investigation of the actions of zinc at the cellular level relating to effects on acid secretion.

Helicobacter pylorus resides within the mucous layer of the human gastric mucosa. Due to extremely low pH, the stomach is a hostile environment to most other microorganisms. The ability of H. pylori to flourish in the stomach has been attributed to protective mechanisms such as its production of urease, protecting the bacterium from gastric acidity by creating a basic microenvironment, See, Taylor and Blaser, Epidemiol Rev, 13:42-59, (1991).

The stomach is a large organ that can be divided into 3 main zones that are involved in the process of digestion of foodstuff and the sterilization of liquids and water. When defining the functional process of the stomach it has been commonly divided into two zones: Upper Stomach, and Lower Stomach. The upper stomach, is thought to be composed of the fundus and upper body, and shows low frequency, sustained contractions that are responsible for generating a basal pressure within the stomach. Of note is that these tonic contractions also generate a pressure gradient from the stomach to small intestine and are responsible for gastric emptying. Interestingly, when swallowing food and the consequent gastric distention that occurs acts to inhibits contraction of this region of the stomach, allowing it to balloon out forming a large reservoir without a significant increase in pressure. The lower stomach is thought to be involved in the grinding and liquefaction of the foodstuffs by the secretion of HCl from the parietal cells found in this section of the stomach.

Zinc salts are now known to be used to increase the pH of gastric juices in a patient or subject in need. Certain zinc salts have been found to be more active than other zinc salts in raising the pH of the gastric juice in that certain zinc salts are more effective and can be used to raise the pH of the gastric juices at a lower concentration or dose.

### BRIEF DESCRIPTION OF THE FIGURES

FIG. 1 shows dose curves for ZincSO4Heptahydrate. The experiments conducted show that at doses from 300 to 0.3 μM zinc sulfate heptahydrate evidences a significant p<0.0001 reduction in secretagogue induced proton secretion (acid production). Zinc sulfate heptahydrate thus acts unexpectedly at very low concentrations to provide efficacy in raising the pH of gastric juices. At a dose of 0.03 μM we still have a p<0.03 significant reduction in secretagogue induced proton secretion. This figure demonstrates the ability to use very low dose while maintaining efficacy for zinc sulfate heptahydrate.

FIG. 2 shows modulation of proton extrusion rates (acid secretion) following exposure to the secretagogue Carbachol (20004) in the presence of various concentrations of ZnSO4.7H₂O in isolated human glands. Note that doses of 300 μM and 3 μM were used following consultation. Both doses in isolated human glands gave a highly significant inhibition of proton extrusion (p<0.0001). All data are the means from 5 separate human samples.

FIG. 3 shows the results of studies on isolated stomachs from 12 h fasted rats (ad lib water access). We see statistically significant fall in intragastric pH following 15 minute exposure to secretagogue on blood side. In paired studies we demonstrate that simultaneous addition of 30 μM ZnSO4Heptahydrate gives a significant elevation in gastric pH of ˜1 pH unit or more.

FIG. 4 shows the results of studies on isolated stomachs from 12 h fasted rats (ad lib water access). We see statistically significant fall in intragastric pH following 15 minute exposure to secretagogue on blood side. In paired studies we demonstrate that simultaneous addition of 3 μM ZnSO4Heptahydrate gives a significant elevation in gastric pH of ˜1 pH unit or more. These results demonstrate that acute exposure to 3 μM (corresponding to a dose of about 0.5 mg in a human patient) is as effective as 30 μM ZnSO4Heptahydrate (corresponding to a dose of about 5 mg in a human patient) in raising the pH of gastric juices.

FIG. 5 demonstrates that there is a memory effect on the isolated glands. At 30 μM ZincSulfate Heptahydrate there is no basal acid secretion in the gland and the addition of the carbachol (secretagogue) has no effect. Demonstrates that there is a memory effect so that if animal has 1 week ad lib access to Zn there is no secretagogue induced acid secretion and the effect lasts for a number of days.

FIG. 6 shows that there is a significant memory effect on tissue exposed to zinc sulfate heptahydrate. Animals were on 3 μM ZincSO4Heptahydrate for 7 days (in drinking water). Matched controls same age sex matched with normal drinking water. The results of this experiment demonstrates that there is a memory effect on the isolated glands. Although basal acid secretion in the gland was slightly higher than blank water, they had a marked suppression in Carbachol (ie Secretagogue) induced acid secretion.

### BRIEF DESCRIPTION OF THE INVENTION

It has now been discovered that at least one zinc salt hydrate, preferably zinc sulfate heptahydrate, used alone or in combination with another zinc salt hydrate and/or a zinc salt non-hydrate is extremely active in raising the pH of the gastric juices in a patient or subject at very low doses, in some cases at doses as small as 1/10^{th}, 1/100^{th} or 1/1000^{th} (30 μM, 3 μM, even 0.3 μM) the dose required using a prior art zinc salt non-hydrate such as zinc chloride, zinc acetate or zinc gluconate, among others. This unexpected activity of the zinc salt hydrates, in particular, zinc sulfate heptahydrate, may be used to raise the pH of the gastric juices of a patient or subject from an acidic level of less than about pH 2.0 to a pH within the range of about 3.0 to about 4.0 (or more) depending upon the patient or subject to whom the presently claimed compositions are administered (i.e, the salt raises the pH of the gastric juices about 1.0 to about 2.5 pH units or more, about 1.0 to about 2.0 pH units, 1.0 to about 1.2 pH units, about 1.0 to about 1.5 pH units, about 1.0 to about 1.75 units) in a very short period of time, often no greater than about 30 minutes, no greater than about 20 minutes, no greater than about 15 minutes, no greater than about 10 minutes at very low doses compared to prior art zinc salt non-hydrates.

Pursuant to the present invention, it has been discovered unexpectedly that zinc salt hydates, especially zinc sulfate heptahydrate are extremely active in raising the pH of gastric juices in a subject and the rise in pH is fast (in as little as 10-15 minutes) and occurs at very low doses of zinc salt hydrate (preferably, zinc sulfate heptahydrate), e.g. at an effective dose as low as 0.03 μM up to about 300 μM or more, often about 0.3 μM to about 300 μM, about 0.3 to about 30 μM, about 1 to about 10 μM, about 1 to about 5 μM, about 0.3 to about 3 μM. These concentrations may be attained using a dose of zinc sulfate heptahydrate and other zinc salt hydrates of about 0.05-50 mg or more, about 0.1 to about 1 mg., about 0.5 to about 5, about 1 mg to about 10 mg, about 2.5 mg to about 7.5 mg, about 10-15 mg., about 15-20 mg., 25 mg., up to about 50 mg or more). A concentration of zinc salt hydrate, especially zinc sulfate heptahydrate in gastric juices of a patient or subject has been found to be particularly effective to raise the pH of the gastric juices of the patient or subject at a concentration of about 1-10 μM, in particular at about 3 μM (ie, at a dose for an adult human ranging from about 0.1 to about 10.0 mg, often about 0.5 mg to about 5 mg., often about 0.5 mg. to about 2.5 mg, about 2.5 mg to about 10 mg). This activity is unexpected from the teachings of the art.

The present invention relates to novel compositions and methods for the rapid inhibition of acid secretion that has little to no potential for side effects. In a first aspect, the present invention relates to compositions comprising a zinc salt hydrate, especially zinc sulfate heptahydrate, alone or in combination only with one or more zinc salt hydrate(s) as the only zinc salt(s) in the composition (in certain aspects of the invention, zinc sulfate heptahydrate will be the only acid lowering agent in the composition) in an effective amount which produces a rapid decrease (i.e., within a period of no greater than about 5 minutes, no greater than about 10 minutes, no greater than about 20 minutes, no greater than about 30 minutes, no greater than one hour) of acid secretion in a patient's stomach with a resulting increase (elevation) in stomach pH from an initial low pH of no greater than about 2.0-2.5 (often between 1.5 and 2.0 or even less) to a subsequent pH (the pH which occurs after the zinc sulfate heptahydrate effects its activity on raising pH of the gastric juices) of at least 1 pH unit higher to about 2 pH units higher than an original acid pH, often about 1.2 pH units higher to about 1.75 pH units higher than the initial acidic Ph (for example, during a patient's or subject's secretogogue phase which produces a low ph in the gastric juices), about 1.0 to about 1.5 pH units higher, to an intragastric pH level of at least about 3.0-4.0, at least about 3.0 to about 3.75, at least about 3.2 to about 3.5 to up to about 4.0 or more.

In one embodiment, a patient or subject in need of an increase of stomach pH is treated with an effective amount of a composition comprising an effective amount of at least one zinc salt hydrate, preferably zinc sulfate heptahydrate, often as the sole zinc salt(s) in the composition, such that rapid onset of elevated pH within the stomach occurs at unexpectedly low doses of zinc salt when the composition containing the salt is administered to the stomach of a patient or subject in need. The present invention relies on the administration (preferably by, but not limited to, oral administration, including ingestion or intubation including oral or nasal intubation) of an effective amount of at least one zinc salt hydrate, often zinc sulfate heptahydrate alone or in combination with at least one additional zinc salt hydrate and/or a pharmaceutically compatible, preferably a water-soluble zinc salt (non-hydrate) in which a substantial portion dissolves in the gastric juices of the patient or subject to be treated at low pH (generally a pH of less than about 2.0-2.5) and often within a range of pH from low pH (about 1.0 to about 2.0) such that effective amounts of zinc salt hydrate, often zinc sulfate heptahydrate may be administered to provide an initial rapid inhibition of acid release, a rapid increase in the pH of the gastric juices and a subsequent maintenance of inhibition of acid release in the stomach. In the case of zinc sulfate heptahydrate, this effect occurs at much lower concentrations/amounts of this salt than for other salts, even at molar concentrations 1/10 or 1/100 of prior art salts exhibiting a similar effect. In the present invention, inhibition of gastric acid occurs rapidly, often within about 10-20 minutes to about 1 hour (in some instances, within a period no greater than about 5 minutes, within a period no greater than about 10 minutes or within a period no greater than about 20 minutes, within a period no greater than about 30 minutes, within a period no greater than about one hour), often depending on how the zinc salt hydrate is delivered to the gastric juices to effect its action. The result is that zinc sulfate heptahydrate can obtain a maximal pH increasing effect of the gastric juices in the stomach of a patient or subject in need at a far lower concentration than is required from other zinc salts, including particularly effective zinc salts such zinc chloride, zinc acetate, zinc gluconate, zinc lactate and zinc picolinate which are non-hydrated.

Compositions according to the present invention which comprise effective amounts of a zinc salt hydrate, preferably zinc sulfate heptahydrate (often, as the only zinc salt within the composition) which are capable of raising the pH from an initial acidic pH (generally the initial pH will range from about 0.8-1.0 to upwards of about 2.0-2.5 or more (in particular, a pH of about 3-4 or more), depending on the patient or subject) at least 1.0 pH unit up to about 2.0 pH units (often at least about 1.2 pH unit up to about 1.75 pH units, at least about 1.25 to about 1.5 pH units) higher than the initial acidic pH in a period of no more than about one hour, often 30 minutes or less, 20 minutes or less or even 10 minutes or less. The zinc salt hydrate, often zinc sulfate heptahydrate, which is included in compositions according to the present invention may be combined with at least one additional zinc salt hydrate or a zinc salt non-hydrate as otherwise described herein and zinc salt hydrate, often zinc sulfate heptahydrate, and optional additional zinc salt(s) hydrates/non-hydrates may be combined with another agent such as an antimicrobial/antibiotic, such as amoxicillin, clarithromycin (biaxin), metronidazole (flagyl) and tetracycline (“an anti-H. pylori agent”); H₂-blockers, such as cimetidine (tagamet), famotidine (pepcid), nizatidine (axid), ranitidine (zantac); proton pump inhibitors (PPI), such as esomeprazole (nexium), lansoprazole (prevacid), omeprazole (prilosec), pantoprazole (protonix) and rabeprazole (aciphex); cytoprotective agents, such as bismuth subsalicylate, sucralfate; combination agents, antacids including bicarbonate (sodium, potassium, calcium, magnesium, etc.), carbonates (sodium, potassium, magnesium and calcium), hydroxide (aluminum and magnesium), bismuth subsalicylate, an effective amount of copper, acetyl salicyclic acid, a blood thinning agent such as arixtra, clopidogrel, coumadin, dipyidamole (alone or with acetyl salicylic acid-asa), fragmin, lovenox, lovenox Hp, plavix, prasugrel and mixtures thereof and mixtures thereof, as otherwise described herein. In preferred aspects of the invention, zinc salt hydrates, especially zinc salt heptahydrate, is the only pH raising component in the composition. In other embodiments, zinc salts including zinc sulfate heptahydrate are the only pH raising component in the composition, the composition avoiding antacids and other such pH increasing components which often cause pH rebound effects subsequent to administration, an unnecessary side effect which could compromise therapy.

Without being limited by way of theory, it is believed that the rapid decrease of acid secretion in the patient's stomach occurs throughout the stomach in both the upper stomach and lower stomach through inhibition of H⁺,K⁺-ATPase, although localized effects of compounds according to the present invention in the upper stomach, especially in the fundic region of the stomach (through inhibition of a second distinguishable protein, H⁺-ATPase) and/or the upper body of the upper stomach (through inhibition of H⁺,K⁺-ATPase). Thus, an additional aspect of the invention is directed to the use of effective amounts of zinc salt hydrate, often zinc sulfate heptahydrate for the inhibition of H⁺,K⁺-ATPase (generally throughout the stomach, H+-ATPase (primarily in the fundic region of the stomach) and preferably both. The finding that the present compounds may be used to inhibit H⁺-ATPase in the fundic region has important clinical ramifications for the following reasons:

1) The erosion of the esophagus by exposure to acid has life threatening consequences due to either internal bleeding, ulceration, and or gastric carcinoid formation by the prolonged exposure to acid. Pursuant to the present invention, as is now demonstrated—glands in the fundus are in direct proximity to the esophageal juncture, that they will secrete acid and can be inhibited by a zinc salt hydrate, often zinc sulfate heptahydrate pursuant to the present invention, thus making the present compounds particularly effective in treating GERD, NERD and related conditions.
2) There is an ever increasing number of patients that are becoming insensitive to PPI's (proton pump inhibitors) and have recurrent symptoms of acid reflux disease. The protein that we identified in the fundic glands is not sensitive to PPI's and could be the reason that these patients do not respond to classical therapy. Some of these patients may be sensitive to higher dose zinc and a lower dose zinc, such as is exhibited by zinc salt hydates, especially zinc sulfate heptahydrate, may be easier for these patients to manage.
3) Patients on PPI's for long periods of time appear to show some “rebound” acid secretion. This result could again be linked to the fundic H⁺-ATPase, which we show is sensitive to histamine and to the levels of protons within the cell. In contrast, the present invention exhibits a memory effect, as described below, lasting several days to a week or more.
4) The use of a zinc salt hydrate, especially zinc sulfate heptahydrate causes a memory effect in that patients who have received the zinc salt hydrate, especially zinc sulfate heptahydrate over a period of therapy will exhibit the same physiological effect for a subsequent period even in the absence of administration of zinc sulfate heptahydrate. This effect, which often occurs after only two or three days of zinc sulfate heptahydrate administration, or a week of administration, or a month of therapy, will often last for at least a day, often several days, often at least 5 days, at least a week or sometimes even more.

Without being limited by way of theory, it is postulated that zinc sulfate hepthydrate or ZnSO4.7H2O comprises the [Zn(H2O)₆]²⁺ hydrated ion when dissolved in aqueous solution and it is the formation of this hydrated species and/or other hydrated species which allows zinc heptahydrate to rapidly dissolve and form a cationic species in the gastric juice which can act so rapidly at such a low concentration compared to other zinc salts, including other hydrated species of zinc salts, to rapidly increase the pH of gastric juices in a patient or subjected administered zinc sulfate heptahydrate. When dissolved, the other H₂O molecule of ZnSO4.7H2O is almost certainly H-bonded to sulfate ions in the lattice, which may provide an additional benefit in producing a rapid increase in the pH of gastric juices.

In certain embodiments of the present invention, at least one zinc salt hydrate, often zinc sulfate heptahydrate may be coadministered with another zinc salt (non-hydrate) which is water-soluble. When another zinc salt is combined with zinc salt hydrate, often zinc sulfate heptahydate, in compositions and/or methods according to the present invention, zinc chloride, zinc acetate and zinc gluconate, zinc lactate and zinc picolinate (non-hydrates) are the preferred salts for use in combination with zinc sulfate heptahydrate in the present invention. It is noted that zinc chloride, zinc acetate, zinc gluconate, zinc lactate and zinc picolinate (in various forms as described herein) are also found as hydrate salts which may be used preferably in combination with zinc sulfate heptahydate when a combination of zinc salt hydrates is used in the present invention. In alternative embodiments, zinc salt hydrate(s), often zinc sulfate heptahydrate may be combined with a mixture of a low pH soluble zinc salt and/or a high pH soluble zinc salt or a zinc salt which may be readily absorbed through the small intestine (such as a zinc amino acid chelate compound), optionally in combination with a pharmaceutically acceptable buffer is provided. In this aspect of the invention, an effective amount of a zinc salt (non-hydrate) selected from the group consisting of zinc chloride (ZnCl₂), zinc acetate, zinc gluconate, zinc ascorbate, zinc citrate, zinc lactate, zinc picolinate, zinc succinate, zinc tartrate, zinc malate, zinc maleate, a zinc amino acid chelate (mono- or bis-chelate) and mixtures thereof, especially a mixture of two or more of zinc chloride, zinc acetate, zinc gluconate, zinc citrate, zinc lactate and zinc picolinate often a mixture of zinc chloride and at least one of zinc acetate, zinc citrate, zinc gluconate, zinc lactate, zinc picolinate, zinc succinate, zinc ascorbate, and a zinc amino acid chelate is provided in combination with zinc hydrate(s), often zinc sulfate heptahydrate, optionally in combination with a pharmaceutically acceptable carrier, additive or excipient.

In various aspects, the present invention relates to the use of zinc salt hydrate(s), including zinc sulfate heptahydrate alone or in combination with at least one additional water-soluble zinc salt (non-hydrate), optionally in combination with at least one compound/composition (within the context of the disease state or condition to be treated) selected from the group consisting of a traditional proton pump inhibitor compound/composition, an antacid, an H2 blocker, an antibiotic/antimicrobial agent (effective against H. pylori), a cytoprotective agent or a mixture of these agents (Helidac, Prevpac) to provide fast action in reducing gastric acid secretion, to lower the pH of the stomach, to prevent or reduce the likelihood of ulcer disease, to treat ulcer disease, to treat gastric cancer, to treat a disease or condition selected from the group consisting of gastroesophageal reflux disease (GERD), non-erosive reflux disease (NERD), Zollinger-Ellison syndrome (ZE disease), ulcer disease, and gastric cancer, as well as preventing or reducing the likelihood of ulcer disease. In certain aspects of the invention, an effective amount of copper (about 100 mcg. to about 10-15 mg. (often, about 500 mcg. to about 1 mg., about 750 mcg.-1 mg to about 5-10 mg) may be administered to a patient at least one hour after zinc salt administration (preferably, at least about 2-8 hours after zinc salt administration) with the zinc salt hydrate in order to reduce the likelihood and/or eliminate a copper deficiency.

Pharmaceutical compositions comprise an effective amount of zinc salt hydrate(s), often zinc sulfate heptahydrate alone or in combination, optionally in further combination with one or more additional zinc salts (non-hydrate). The zinc salt hydrate(s) (often zinc sulfate heptahydrate alone or in combination with another zinc salt hydrate) is preferably administered in immediate release form and other zinc salts which may be optionally combined with the zinc salt hydrate(s) and formulated to maximize both immediate and extended release characteristics of the present invention, optionally in combination with a pharmaceutically acceptable carrier, additive or excipient. These compositions may be formulated alone or optionally in combination with an effective amount of additional agent selected from the group consisting of a proton pump inhibitor, an H₂ blocker, an anti-H. pylori antibiotic/antimicrobial, a cytoprotective agent and a combination of these agents. Any one or more of these compositions may be used within context to treat the various conditions/disease states as otherwise disclosed herein. It is noted for example, that the zinc salt hydrate(s), especially zinc sulfate heptahydrate, is preferably used in immediate dosage form to take advantage of its unexpected rapid activity in raising the pH of gastric juices at very low dosages (compared to other zinc salts) and at least one additional zinc salt (non-hydrate) may be used to provide an additional immediate release effect or in preferred embodiments, an extended release effect. Additionally, in compositions according to the present invention, an effective amount of an additional agent selected from the group consisting of a proton pump inhibitor, an H₂ blocker, an anti-H. pylori antibiotic/antimicrobial, a cytoprotective agent or a combination of agents may be used in immediate and/or extended release form. In one aspect of the invention, the zinc salt hydrate (preferably, zinc sulfate heptahydrate) may be formulated in combination with an additional agent as described above in immediate dose form and/or extended dose form. In certain instances, because of the rapid increase in pH of the gastric juices which is produced by the administration of zinc salt hydrate (preferably, zinc sulfate heptahydrate), numerous agents which may be unstable to stomach acid can be administered in immediate release form, thus substantially enhancing the bioavailability of these agents. Many of these agents are identified and listed in the specification as provided below.

In an additional aspect of the present invention, pharmaceutical compositions comprise an effective amount of at least one zinc salt hydrate, preferably at least zinc sulfate heptahydrate, alone, in combination with another zinc salt hydrate and/or in combination with at least one additional water soluble zinc salt (non-hydrate) as otherwise described herein in combination with at least one (additional) therapeutic agent wherein the oral administration of said agent is favorably affected by elevated pH levels in the stomach, optionally in combination with a pharmaceutically acceptable carrier, additive or excipient. It has been discovered that the inclusion of zinc salt hydrate(s), in particular, zinc sulfate heptahydrate alone or in combination with another zinc salt hydrate and optionally, an additional water soluble zinc salt (non-hydrate) as otherwise described herein to rapidly raise the pH of the gastric juices, may be used in combination with a therapeutic agent which is favorably responsive to elevated pH levels in the stomach of a patient or subject (because of the tendency of the agent to be unstable to or to produce/increase undesired acidity in the stomach), to substantially enhance the stability and/or the bioavailability of the therapeutic agent. This approach obviates the acid sensitivity of the therapeutic agent, enhances solubility for those agents which are more soluble at higher acid′pH's (the majority) at a pH of about 3.0-4.0 or higher, and/or because of the tendency of the therapeutic agent to create GI tract distress or ulcerations at lower pH's, which are substantially reduced or alleviated at higher pH's. Thus, the combination of zinc salt hydrate(s), especially zinc sulfate heptahydrate, which is used as the sole zinc salt, combined with another zinc salt hydrate or combined with another zinc salt (non-hydrate) to enhance the oral administration of therapeutic agents (generally, advantageously in immediate release formulations to minimize the impact of gastric acid on the agent coadministered with the zinc salt hydrate, preferably zinc sulfate heptahydrate) by increasing their bioavailability and/or decreasing the side effects from the therapeutic agent represent additional embodiments of the present invention.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “patient” or “subject” refers to an animal, preferably a domesticated animal, preferably a mammal, even more preferably a human, in need of treatment or therapy to which compounds according to the present invention are administered in effective amounts in order to treat a condition or disease state treatable using compounds according to the present invention. Depending upon the disease or condition treated. the term patient refers to the animal treated for that disease within context. The present invention contemplates administration to humans and to domesticated animals, including dogs, cats and related pets, among others and cows, horses, pigs and related farm animals, among others.

The term “effective” is used to describe a treatment, compound, composition, component or a related aspect of the present invention, which, when used in context, produces an intended result which may include the increase in pH in the stomach, the reduction of symptoms associated with excess acid release, the enhanced bioavailability of an administered compound, or the favorable treatment of a disease state or condition. The term effective subsumes both an amount or concentration of one or more active agent(s) as described herein and a period of time which is consistent with producing an intended effect.

The term “zinc salt hydrate(s)” refers to a single zinc salt hydrate or a combination of zinc salt hydrates, which especially includes the zinc salt hydrate, zinc sulfate hepathydrate. Preferred zinc salt hydrate(s) for use in the present invention include the following zinc salt hydrates, which may be used as mixtures: zinc chloride monohydrate, zinc chloride dehydrate, zinc chloride trihydrate, zinc chloride quatrahydrate, zinc acetate dihydrate, zinc citrate dihydrate, zinc citrate trihydrate, zinc gluconate monohydrate, zinc gluconate dihydrate, zinc gluconate trihydrate, zinc bromide dihydrate, zinc fluoride dihydrate, zinc lactate monohydrate, zinc lactate dehydrate, zinc lactate trihydrate, zinc nitrate hydrate, zinc perchlorate hexahydrate, zinc picolinate dehydrate, zinc sulfate monohydrate, zinc tetrafluoroborate hydrate, zinc p-toluenesulfate hydrate and mixtures thereof. Preferred zinc salt hydrates for use in the present invention include zinc chloride monohydrate, zinc chloride dihydrate, zinc chloride trihydrate, zinc chloride quatrahydrate, zinc acetate dihydrate, zinc gluconate monohydrate, zinc gluconate dihydrate, zinc gluconate trihydrate, zinc citrate dihydrate, zinc citrate trihydrate, zinc lactate monohydrate, zinc lactate dehydrate, zinc lactate trihydrate, zinc picolinate dihydrate. Zinc sulfate heptahydrate is a preferred zinc salt hydrate for use in the present invention.

The term “additional pharmaceutically acceptable zinc salt” or additional zinc salt” used in context, refers to a salt or salt combination other than at least one zinc salt hydrate, in particular, zinc sulfate heptahydrate which contains zinc, dissolves in the gastric juices at reduced pH and is absorbed to some extent in the gastric mucosa at a low pH of about 2-3 or less, at a higher pH of about 4.0 to 5.0 or above of the stomach and at the high pH's of the small intestine to reach and maintain effective concentrations of zinc in the blood stream over a period of therapy. Exemplary pharmaceutically compatible zinc salts (non-hydrates or anhydrous) which may, in certain embodiments, be combined with one or more zinc salt hydrates, including zinc sulfate heptahydrate include both inorganic and organic zinc salts, for example, zinc acetate, zinc ascorbate, zinc benzoate, zinc bromide, zinc butyrate, zinc caprylate, zinc carbonate (soluble in dilute acid at low pH of the stomach), zinc carnosine, zinc citrate, zinc chloride, zinc fluoride, zinc formate, zinc fumarate, zinc fumaric acid monoethyl ester, zinc gallate, zinc gluconate, zinc glutarate, zinc glycerate, zinc glycerophosphate, zinc glycolate, zinc hydroxide, zinc iodide, zinc iodate, zinc lactate, zinc malate, zinc maleate, zinc myristate, zinc nitrate, zinc oratate, zinc oxide, zinc phenol sulfonate, zinc phosphate, zinc picolinate, zinc picrate, zinc propionate, zinc salicylate, zinc selenate, zinc stearate, zinc succinate, zinc sulfate, zinc tannate, zinc tartrate, zinc undecylenate, zinc valerate, and zinc chelates, including zinc amino acid chelates (including, depending on concentration, mono- and bis-chelates of L- or D-amino acids (preferably, the naturally occurring L-amino acid which may be more readily absorbed from the gastrointestinal tract) which complex or chelate with zinc including preferably, L-arginine (zinc arginate), L-cysteine, L-cystine, L-N-acetylcysteine, L-histidine (also D-histidine as zinc histidinate), L-taurine, L-glycinate, L-aspartate (zinc aspartate) and L-methionine (zinc methionine), among others. Note that for purposes of the present invention, zinc chelates, including zinc nicotinamide complex and zinc amino acid chelates are considered zinc salts. Preferred zinc salts for use in the present invention include zinc acetate, zinc arginate, zinc butyrate, zinc chloride, zinc citrate, zinc formate, zinc fumarate, zinc gluconate, zinc glutarate, zinc glycerate, zinc glycolate, zinc histidinate, zinc lactate, zinc malate, zinc maleate, zinc picolinate, zinc propionate, zinc salicylate, zinc succinate, zinc sulfate, zinc undecylenate, zinc salt of 1,6 fluctose diphosphate and mixtures thereof. Especially preferred zinc salts including zinc chloride, zinc acetate and zinc gluconate.

Preferably, the pharmaceutically acceptable zinc salt is “water soluble”. The term “water soluble” is used to describe a zinc salt (and zinc chelates which fall under this term) according to the present invention which has a water solubility of at least about 0.01 moles/Liter, preferably at least about 0.05 moles/Liter.

One of ordinary skill will recognize favorable zinc salts to use in combination with the zinc salt hydrate(s), especially zinc sulfate heptahydrate in compositions according to the present invention. In aspects of the invention, an effective amount of zinc salt hydrate, especially zinc sulfate heptahydate alone or in combination with at least one additional zinc salt hydrate is optionally combined with at least one pharmaceutically compatible, water-soluble zinc salt non-hydrate and administered to a patient in order to provide a rapid inhibition of acid release in the stomach, resulting in an increase in stomach pH to above 2.5-3.0 up to about 4 (generally between about 3.0 and 4.0, in some cases above 4.0) for an extended period of time, preferably at least 2 hours, 4 hours, 6 hours, 8 hours, 10 hours, 12 hours, 16 hours, 20 hours or more. It is noted that in certain preferred aspects of the invention, the zinc salt hydrate or combination of zinc salt (hydrates(s) and optionally non-hydrates chosen to be administered to the patient may be adjusted to provide an initial bolus concentration of zinc in the stomach at low pH in order to produce the rapid inhibition of acid release and rise in pH in the stomach to a level above about 3-3.5 and up to about 4.0 and above. In addition, a preferred zinc salt combination inhibits acid release in the stomach at varying levels of acidity and pH—i.e., at a level which is quit acidic (pH, less than about 2.0-2.5) to a pH of about 4.0 or higher.

The term “providing fast action in reducing gastric acid secretion” is used to describe the fact that the method according to the present invention results in an increase in pH of at least 1.0 pH unit up to about 2.0 pH units or more, about 1.25 pH units to about 1.75 pH units, about 1.25 units up to about 1.5 pH units, to a pH level of at least about 3.0-3.5, often about 3.5 to 4.0 or slightly above, in a period of no greater than one hour, often no great than about 30 minutes, preferably in less than about 20-30 minutes, even more preferably in less than about 10-20 minutes, in about 15 minutes or less or alternatively, in less than about 5 minutes. This will depend upon the dissolution profile of the dosage form of zinc sulfate heptahydrate in gastic juice.

The term “secretagogue” refers to the period during which time the pariental cells of the stomach secrete acid into the gastric juices to lower pH. Often the secretagogue period occurs just after a meal, but the secretion of acid may occur at other times. The secretagogue phase can be of short duration or longer duration. The initial pH at which the present compositions are used is often during the secretagogue period. The present method and compositions are effective at raising the pH of gastric juices especially during or subsequent to secretagogue, among other times, when acid is released.

The term gastroesophageal reflux disease or “GERD” or “acid reflux” is a condition in which the liquid content of the stomach regurgitates (backs up, or refluxes) into the esophagus. The liquid can inflame and damage the lining of the esophagus although this occurs in a minority of patients. The regurgitated liquid usually contains acid and pepsin that are produced by the stomach. The refluxed liquid also may contain bile that has backed-up into the stomach from the duodenum. Acid is believed to be the most injurious component of the refluxed liquid. Pepsin and bile also may injure the esophagus, but their role in the production of esophageal inflammation and damage (esophagitis) is not as clear as is the role of acid.

GERD is a chronic condition. Once it begins, it usually is life-long. If there is injury to the lining of the esophagus (esophagitis), this also is a chronic condition. Moreover, after the esophagus has healed with treatment and treatment is stopped, the injury will return in most patients within a few months. Once treatment for GERD is begun, therefore, it may be necessary to continue the treatment continually, generally for short periods of time.

Actually, the reflux of the stomach's liquid contents into the esophagus occurs in most normal individuals. In fact, one study found that reflux occurs as frequently in normal individuals as in patients with GERD. In patients with GERD, however, the refluxed liquid contains acid more often, and the acid remains in the esophagus longer.

Gravity, swallowing, and saliva are important protective mechanisms for the esophagus, but they are effective only when individuals are in the upright position. At night while sleeping, gravity is not in effect, swallowing stops, and the secretion of saliva is reduced. Therefore, reflux that occurs at night is more likely to result in acid remaining in the esophagus longer and causing greater damage to the esophagus.

Certain conditions make a person susceptible to GERD. For example, reflux can be a serious problem during pregnancy. The elevated hormone levels of pregnancy probably cause reflux by lowering the pressure in the lower esophageal sphincter (see below). At the same time, the growing fetus increases the pressure in the abdomen. Both of these effects would be expected to increase reflux. Also, patients with diseases that weaken the esophageal muscles (see below), such as scleroderma or mixed connective tissue diseases, are more prone to develop reflux.

The cause of GERD is complex. There probably are multiple causes, and different causes may be operative in different individuals or even in the same individual at various times. A number of patients with GERD produce abnormally large amounts of acid, but this is uncommon and not a contributing factor in the vast majority of patients. The factors that contribute to causing GERD are the lower esophageal sphincter, hiatal hernias, esophageal contractions, and emptying of the stomach. Notwithstanding the cause of GERD, the present invention may reduce the tendency of having injurious acid reflux into the esophagus, causing damage.

When the wave of contraction in the esophagus is defective, refluxed acid is not pushed back into the stomach. In patients with GERD, several abnormalities of contraction have been described. For example, waves of contraction may not begin after each swallow or the waves of contraction may die out before they reach the stomach. Also, the pressure generated by the contractions may be too weak to push the acid back into the stomach. Such abnormalities of contraction, which reduce the clearance of acid from the esophagus, are found frequently in patients with GERD. In fact, they are found most frequently in those patients with the most severe GERD. The effects of abnormal esophageal contractions would be expected to be worse at night when gravity is not helping to return refluxed acid to the stomach. Note that smoking also substantially reduces the clearance of acid from the esophagus. This effect continues for at least 6 hours after the last cigarette.

Most reflux during the day occurs after meals. This reflux probably is due to transient LES relaxations that are caused by distention of the stomach with food. A minority of patients with GERD, about 20%, has been found to have stomachs that empty abnormally slowly after a meal. The slower emptying of the stomach prolongs the distention of the stomach with food after meals. Therefore, the slower emptying prolongs the period of time during which reflux is more likely to occur.

The term “non-erosive reflux disease” or “NERD” is used describe a specific form of GERD, described above. In some cases, GERD erodes the esophageal lining, creating a condition called esophagitis. NERD is GERD that does not cause esophagitis. Because most GERD sufferers do not have esophagitis, NERD is the most common form of GERD. Because its name contains the word “nonerosive,” it may appear that NERD is the least severe form of GERD, but this is not necessarily so. NERD is actually more likely to produce extra-esophageal complications, and is also less likely to respond to fundoplication surgery. In one study, only 56% of NERD patients (compared with 90% of patients with erosive reflux) reported that their symptoms were completely eliminated with fundoplication. NERD was also twice as likely to cause swallowing difficulties.

Heartburn is the chief symptom of NERD. It has a number of potential causes, including hiatal hernia, lifestyle behaviors, and diet. Many people deal with heartburn by simply adjusting their behavior. In some cases, medication or surgery may be required. Traditional antacids have also been used to treat NERD.

The term “Zollinger-Ellison syndrome” or “ZE syndrome” is used throughout the specification to describe a condition caused by abnormal production of the hormone gastrin. In ZE syndrome, small tumor (gastinoma) in the pancreas or small intestine produces the high levels of gastrin in the blood. ZE syndrome is caused by tumors usually found in the head of the pancreas and the upper small bowel. These tumors produce the hormone gastrin and are called gastrinomas. High levels of gastrin cause overproduction of stomach acid. High stomach acid levels lead to multiple ulcers in the stomach and small bowel. Patients with ZE syndrome may experience abdominal pain and diarrhea. The diagnosis is also suspected in patients without symptoms who have severe ulceration of the stomach and small bowel.

The agents of choice for treating ZE syndrome are the proton pump inhibitors (PPI) as described hereinabove. These drugs dramatically reduce acid production by the stomach, and promote healing of ulcers in the stomach and small bowel. They also provide relief of abdominal pain and diarrhea.

Surgical removal of a single gastrinoma may be attempted if there is no evidence that it has spread to other organs (such as lymph nodes or the liver). Surgery on the stomach (gastrectomy) to control acid production is rarely necessary today. Early diagnosis and surgical removal of the tumor is associated with a cure rate of only 20% to 25%. However, gastrinomas grow slowly, and patients may live for many years after the tumor is discovered. Acid-suppressing medications are very effective at controlling the symptoms of acid overproduction.

The term “ulcer” is used throughout the specification to describe an area of tissue erosion, for example, especially of the lining of the gastrointestinal (GI) tract, especially of the stomach (peptic ulcer), esophagus or small intestine (duodenal ulcer). Due to the erosion, an ulcer is concave. It is always depressed below the level of the surrounding tissue. Ulcers can have diverse causes, but in the GI tract, they are believed to be primarily due to infection with the bacteria H. pyloridus (h. pylori). GI ulcers, however, may be made worse by stress, smoking and other noninfectious factors, especially including excessive stomach acid because a lower pH tends to be a better growth environment for H. Pyloridus.

Traditional treatments for H. pyloridus infections include antimicrobials/antibiotics, such as amoxicillin, clarithromycin (biaxin), metronidazole (flagyl) and tetracycline (“an anti-H. pylori agent”); H₂-blockers, such as cimetidine (tagamet), famotidine (pepcid), nizatidine (axid), ranitidine (zantac); proton pump inhibitors (PPI), such as esomeprazole (nexium), lansoprazole (prevacid), omeprazole (prilosec), pantoprazole (protonix) and rabeprazole (aciphex); cytoprotective agents, such as bismuth subsalicylate, sucralfate; and combination agents, such as Helidac (bismuth subsalicylate, metronidazole, and tetracycline combination), Prevpac (lansoprazole, clarithromycin and amoxicillin).

The present invention may be used to treat an H. pyloridus infection in a patient by administering an effective amount of zinc salt hydrate(s), preferably zinc sulfate heptahydrate, either alone or in combination with another zinc salt hydrate, and optionally, at least one pharmaceutically acceptable water-soluble zinc salt non-hydrate, or in further combination (preferably, by coadministration) with at least one other of the traditional treatment modalities, as described above.

The term “coadministration” or “combination therapy” is used to describe a therapy in which at least two active compounds in effective amounts are used to treat one or more of the disease states or conditions as otherwise described herein at the same time. Although the term coadministration preferably includes the administration of two active compounds to the patient at the same time, it is not necessary that the compounds be administered to the patient at the same time, although effective amounts of the individual compounds will be present in the patient at the same time. The active compositions may include zinc salt hydrate(s), preferably zinc sulfate heptahydrate, either alone or in combination with another zinc salt hydrate, and optionally, at least one pharmaceutically acceptable water-soluble zinc salt non-hydrate, and/or additional compounds/compositions such as proton pump inhibitors, antacids (traditional acid neutralizing agents, although antacids are typically avoided in preferred aspects to reduce the likelihood of side effects from acid rebound which often occurs with these agents), H₂ blockers, antibiotics/antimicrobial agents, cytoprotective agents, combination agents, and mixture thereof as otherwise described herein in effective amounts for the disease or condition for which the compounds are typically used. In addition, coadministration also contemplates combinations of zinc salt hydrate(s), preferably zinc sulfate heptahydrate, either alone or in combination with another zinc salt hydrate, and optionally, at least one pharmaceutically acceptable water-soluble zinc salt non-hydrate, as otherwise described herein in combination with at least one therapeutic agent wherein elevated pH levels provide a favorable response to the administration of said therapeutic agent. In certain aspects, the administration of a copper salt sometime after administration of the zinc containing compositions described herein is useful to reduce the copper reduction which may occur from the administration of zinc salts. Effective amounts of copper salts are often administered at least one hour after the administration of a zinc salt composition, especially including those which are described herein and preferably, copper salts are administered at times of about 1, 2, 3, 4, 5, 6, 7, 8 or more hours after zinc salt administration.

The term “favorably responsive to elevated pH levels” or “favorably orally administered” is used to describe therapeutic agents which, in orally administered compositions, provide a favorable response to an elevated pH in the stomach produced by zinc salt hydrate(s), preferably zinc sulfate heptahydrate, either alone or in combination with another zinc salt hydrate, and optionally, at least one pharmaceutically acceptable water-soluble zinc salt non-hydrate, according to the present invention, whether that favorable response is a reduction in gastric irritation from the therapeutic agent, a reduction in acid generation/production in the stomach by the therapeutic agent, to increase bioavailability which is negatively impacted by the sensitivity and/or inactivation of the therapeutic agent to an acidic environment or because of increased solubility of the therapeutic agent in gastric juices at high pH levels or another favorable benefit. It has been discovered that the inclusion of zinc salt hydrate(s), preferably zinc sulfate heptahydrate, either alone or in combination with another zinc salt hydrate, and optionally, at least one pharmaceutically acceptable water-soluble zinc salt non-hydrate, as otherwise described herein, further in combination with a therapeutic agent which is favorably responsive to elevated pH levels because of the tendency of the agent to increase acid release and a lowering of pH in the stomach, because of increased acid sensitivity of the therapeutic agent, because of enhanced solubility of the agent (with concombinant increased bioavailability of the therapeutic agent) at higher (less acid) pH's of about 3.0-4.0 or higher, and/or because of the tendency of the therapeutic agent to create GI tract distress or ulcerations at lower pH's, which are substantially reduced or alleviated at higher pH's results in greater activity and/or fewer side effects from the therapeutic agent. In some embodiments, compositions may contain one or more zinc salt hydrates (and optionally, additional zinc salts) and at least one therapeutic agent in immediate release form, which may further include additional therapeutic agents in immediate and/or sustained and/or controlled release form (e.g. by formulating a core comprising a sustained or controlled release form of a therapeutic agent surrounded by an immediate release coating which comprises the zinc salt hydrate(s) (and optional zinc salt) and therapeutic agent(s) to be immediately released in the stomach of the patient or subject to whom the composition is administered. Any agent which is absorbed in appreciable amounts in the stomach or duodenum of a patient may be advantageously administered in immediate release form in the stomach of a patient. However, the release of the therapeutic agent in the stomach of the patient may result in the exposure of that therapeutic agent to an acidic environment to which the agent is not stable, resulting in a diminution of active therapeutic agent to be absorbed by the patient. Thus, any therapeutic agent which could be advantageously delivered in immediate release form but may be unfavorably impacted by an acidic environment (“an acid sensitive therapeutic agent”) is a candidate for inclusion in compositions according to the present invention which comprise both a zinc salt hydrate (especially zinc sulfate heptahydrate) and an acid sensitive therapeutic agent

In general, the weight ratio of zinc salt hydrate(s), preferably zinc sulfate heptahydrate, either alone or in combination with another zinc salt hydrate, and optionally, at least one pharmaceutically acceptable water-soluble zinc salt non-hydrate, to therapeutic agent which is included in combination therapeutic compositions according to the present invention ranges from about 1:1000 to about 1000:1, about 1:100 to about 100:1, about 1:50 to about 50:1, about 1:20 to about 20:1, about 1:10 to about 10:1, about 1:5 to about 5:1, about 1:3 to about 3:1, about 1:2 to about 2:1, about 1:1.5 to about 1.5 to 1, about 1:1. Of course, the weight ratio used in a particular combination pharmaceutical composition will depend upon the water solubility of zinc salt hydrate, especially zinc sulfate heptahydrate and any other zinc salt which is combined with the zinc sulfate heptahydrate and the activity of the therapeutic agent in producing a side effect, e.g., such as increasing stomach acid or increasing gastrointestinal distress (GI tract distress) or the tendency to increase the environment for ulceration in the gastrointestinal tract), or to be inactivated, rendered insoluble or have its bioavailability negatively impacted by stomach acid, etc.

Therapeutic compounds which may be favorably administered orally (for the reasons which are outlined above) in combination with a zinc salt hydrate(s), preferably zinc sulfate heptahydrate, either alone or in combination with another zinc salt hydrate, and optionally, at least one pharmaceutically acceptable water-soluble zinc salt non-hydrate, in the present invention include any therapeutic agent which exhibits acid instability and could benefit from immediate release in the stomach of a patient or subject to enhance bioavailability (area under the curve or AUC) of the therapeutic agent and includes the following:

Chemotherapeutic Agents

Zn plus chemolitic agents for use in treating intestinal cancer

e.g., Cisplatin, carboplatin;

Zn plus chemolitic agents used to treat whole tissue cancer with secondary complications in the intestinal track (GI tract distress) as follows:

13-cis-Retinoic Acid;

2-CdA (2-Chlorodeoxyadenosine);

5-Azacitidine;

5-Fluorouracil (5-FU);

6-Mercaptopurine (6-MP);

6-TG (6-Thioguanine);

Abraxane;

Accutane® (Isotretinoin);

Actinomycin-D;

Adriamycin® (Doxorubicin Hydrochloride);

Adrucil® (Fluorouracil);

Agrylin® (Anagrelide;

Ala-Cort® (Hydrocortisone);

Aldesleukin;

Alemtuzumab;

ALIMTA (Pemetrexed);

Alitretinoin;

Alkaban-AQ® (Vinblastine);

Alkeran® (Melphalan);

All-transretinoic Acid;

Alpha Interferon;

Altretamine;

Amethopterin;

Amifostine;

Aminoglutethimide;

Anagrelide;

Kidrolase® (Asparaginase);

Lanacort® (Hydrocortone Phosphate);

L-asparaginase;

LCR (Leurocristine);

Lenalidomide;

Letrozole;

Leucovorin;

Leukeran;

Leukine™ (Sargramostim);

Leuprolide;

Leurocristine;

Leustatin™ (Cladribin);

Liposomal Ara-C;

Liquid Pred® (Deltasone);

Lomustine;

L-PAM (L-phenylalanine mustard, phenylalanine mustard);

L-Sarcolysin;

Lupron® (Leuprolide Acetate Inj);

Lupron Depot® (Leuploride Acetate);

Matulane® (Procarbazine);

Maxidex;

Mechlorethamine;

Mechlorethamine Hydrochloride;

Medralone® (Methylprednisolone);

Medrol® (Methylprednisolone);

Megace® (Megestrol Acetate);

Megestrol;

Megestrol Acetate;

Melphalan;

Mercaptopurine;

Mesna;

Mesnex™ (Mesna);

Methotrexate;

Anandron® (Nilutamide);

Anastrozole

Arabinosylcytosine;

Ara-C;

Aranesp® (Darbepoetin Alfa);

Aredia® (Pamidronate);

Arimidex® (Anastrozole);

Aromasin® (Exemestane);

Arranon® (Nelarabine);

Arsenic Trioxide;

Asparaginase;

ATRA (Atragen);

Avastin® (Bevacizumab);

Azacitidine;

BCG (Bacillus Calmette Guerin);

BCNU (Carmustine);

Bevacizumab;

Bexarotene;

BEXXAR® (Tositumomab and Iodine 1131 Tositumomab);

Bicalutamide;

BiCNU (CARMUSTINE);

Blenoxane® (Bleomycin Sulfate);

Bleomycin;

Bortezomib;

Busulfan;

Busulfex® (Busuflan);

C225 (Eribitux);

Calcium Leucovorin;

Campath® (Alemtuzumab;

Camptosar® (Irinotecan hydrochloride);

Camptothecin-11;

Capecitabine;

Carac™ (Fluorouracil);

50 Carboplatin;

Carmustine;

Carmustine Wafer;

Casodex® (Bicalutamide);

CC-5013 (Revlimid);

CCNU (lomustine);

CDDP (Cisplatin);

CeeNU;

Cerubidine® (Daunorubicin);

Cetuximab;

Chlorambucil;

Cisplatin;

Methotrexate Sodium;

Methylprednisolone;

Meticorten® (prednisone);

Mitomycin;

Mitomycin-C;

Mitoxantrone;

M-Prednisol® (Methlyprednisolone);

MTC (Mitomycin);

MTX (Methotrexate);

Mustargen® (Mechlorethamine HCl);

Mustine;

Mutamycin® (Mitomycin);

Myleran® (Busulfan);

Mylocel™ (Hydroxyurea);

Mylotarg® (Gemtuzumab Ozogamicin);

Navelbine® (Vinorelbine Tartrate);

Nelarabine

Neosar® (Cyclophosphamide);

Neulasta™ (Pegfilgrastim);

Neumega® (Oprelvekin);

Neupogen® (Filgrastim);

Nexavar® (Sorafenib);

Nilandron® (Nilutamide);

Nilutamide;

Nipent® (Pentostatin);

Nitrogen Mustard;

Novaldex® (Genox);

Novantrone® (Mitoxantrone);

Octreotide;

Octreotide acetate;

Oncospar® (Pegylated asparaginase);

Oncovin® (Vincristine Sulfate);

Ontak® (Denileukin Diftitox);

Onxal™ (Paclitaxel);

Oprevelkin;

Orapred® (Prednisolone Sodium Phosphate);

Orasone® (prednisone);

Oxaliplatin;

Paclitaxel;

Paclitaxel Protein-bound;

Pamidronate;

Panitumumab;

Panretin® (Alitretinoin);

Paraplatin® (Paraplatin);

Citrovorum Factor;

Cladribine;

Cortisone;

Cosmegen® (Dactinomycin);

CPT-11 (Topotecan);

Cyclophosphamide;

Cytadren® (Aminoglutethimide);

Cytarabine;

Cytarabine Liposomal;

Cytosar-U® (Cytarabine);

Cytoxan® (Cyclophosphamide);

Dacarbazine;

Dacogen;

Dactinomycin;

Darbepoetin Alfa;

Dasatinib;

Daunomycin;

Daunorubicin;

Daunorubicin Hydrochloride;

Daunorubicin Liposomal;

DaunoXome® (Daunorubicin Liposoma);

Decadron;

Decitabine;

Delta-Cortef® (Prednisolone);

Deltasone® (Prednisone);

Denileukin diftitox;

DepoCyt™ (Cytarabine liposome injection);

Dexamethasone;

Dexamethasone acetate;

Dexamethasone Sodium Phosphate;

Dexasone;

Dexrazoxane;

DHAD (Novantrone);

DIC (Disseminated intravascular coagulation);

Diodex;

Docetaxel;

Doxil® (Doxorubicin HCl liposome);

Doxorubicin;

Doxorubicin liposomal;

Droxia™ (Hydroxyurea);

DTIC (Dacarbazine);

DTIC-Dome® (dacarbazine);

Duralone®;

Efudex® (fluorouracil topical);

Eligard™ (Leuprolide Acetate);

Pediapred® (Prednisolone Sodium);

PEG Interferon;

Pegaspargase;

Pegfilgrastim;

PEG-INTRON™ (Peginterferon alfa-2b);

PEG-L-asparaginase;

PEMETREXED;

Pentostatin;

Phenylalanine Mustard;

Platinol® (Cisplatin);

Platinol-AQ® (Cisplatin);

Prednisolone;

Prednisone;

Prelone® (Prednisolone);

Procarbazine;

PROCRIT® (Epoetin Alfa);

Proleukin® (Aldesleukin);

Prolifeprospan 20 with Carmustine;

Implant;

Purinethol® (Mercaptopurine);

Raloxifene;

Revlimid® (Lenalidomide);

Rheumatrex® (Trexall);

Rituxan® (Rituximab);

Rituximab;

Roferon-A® (Interferon Alfa-2a);

Rubex® (adriamycin)

Rubidomycin hydrochloride;

Sandostatin® (Octreotide Acetate);

Sandostatin LAR® (Octreotide Acetate inj);

Sargramostim;

Solu-Cortef® (Hydrocortisone Sodium Succinate);

Solu-Medrol® (Methylprednisolone sodium succinate);

Sorafenib;

SPRYCEL™ (Dasatinib);

STI-571 (Gleevec);

Streptozocin;

SU11248;

Sunitinib;

Sutent® (Sunitinib Malate);

Tamoxifen;

Tarceva® (Erlotinib);

Targretin® (Bexarotene);

Taxol® (Paclitaxel);

Ellence™ (Epirubicin hydrochloride);

Eloxatin™ (Oxaliplatin Inj);

Elspar® (Asparaginase);

Emcyt® (Estramustine);

Epirubicin;

Epoetin alfa;

Erbitux™ (Cetuximab);

Erlotinib;

Erwinia L-asparaginase;

Estramustine;

Ethyol;

Etopophos® (Etoposide Phosphate);

Etoposide;

Etoposide Phosphate;

Eulexin® (Flutamide);

Evista® (Raloxifene);

Exemestane;

Fareston® (Toremifene);

Faslodex® (Fulvestrant);

Femara® (Letrozole);

Filgrastim;

Floxuridine;

Fludara® (Fludarabine);

Fludarabine;

Fluoroplex® (Fluorouracil topical);

Fluorouracil;

Fluorouracil (cream);

Fluoxymesterone;

Flutamide;

Folinic Acid;

FUDR® (Floxuridine);

Fulvestrant;

G-CSF (Neupogen);

Gefitinib;

Gemcitabine;

Gemtuzumab ozogamicin;

Gemzar® (Gemcitabine);

Gleevec™;

Gliadel® (Carmustine Wafer);

GM-CSF;

Goserelin;

Granulocyte-Colony Stimulating Factor;

Granulocyte Macrophage Colony Stimulating Factor;

Taxotere® (Docetaxel);

Temodar® (Temozolomide);

Temozolomide;

Teniposide;

TESPA (Thiotepa);

Thalidomide;

Thalomid® (Thalidomide);

TheraCys® (Intravesical);

Thioguanine;

Thioguanine Tabloid® (Thioguanine);

Thiophosphoamide;

Thioplex® (Thiotepa);

Thiotepa;

TICE® (Bacillus of Calmette and Guerin);

Toposar® (Etoposide);

Topotecan;

Toremifene;

Tositumomab;

Trastuzumab;

Tretinoin;

Trexall™ (Methotrexate);

Trisenox® (Arsenic);

TSPA (Thiotepa);

VCR;

Vectibix™ (Panitumumab);

Velban® (Vinblastine Sulfate);

Velcade® (Bortezomib);

VePesid® (Etoposide);

Vesanoid® (Tretinoin);

Viadur™ (Leuprolide Acetate Implant);

Vidaza® (Azacitidine);

Vinblastine;

Vinblastine Sulfate;

Vincasar Pfs® (Vincristine Sulfate Injection);

Vincristine;

Vinorelbine;

Vinorelbine tartrate;

VLB (Vinblastine Sulfate);

VM-26;

Vorinostat;

VP-16 (Etoposide);

Vumon® (Teniposide);

Xeloda® (Capecitabine);

Zanosar® (Streptozocin);

Halotestin® (Fluoxymesterone);

Herceptin® (Trastuzumab);

Hexadrol;

Hexalen® (Altretamin);

Hexamethylmelamine;

HMM (antineoplastic or cytotoxic);

Hycamtin® (Hycamtin);

Hydrea® (Hydroxyurea);

Hydrocort Acetate® (Hydrocortisone);

Hydrocortisone;

Hydrocortisone Sodium Phosphate;

Hydrocortisone Sodium Succinate;

Hydrocortone Phosphate;

Hydroxyurea;

Ibritumomab;

Ibritumomab Tiuxetan;

Idamycin® (Idarubicin);

Idarubicin;

Ifex® (Ifosfamide);

IFN-alpha;

Ifosfamide;

IL-11;

IL-2;

Imatinib mesylate;

Imidazole Carboxamide;

Interferon alfa;

Interferon Alfa-2b (PEG Conjugate);

Interleukin-2;

Interleukin-11;

Intron A® (interferon alfa-2b);

Iressa® (Getfitinib);

Irinotecan;

Isotretinoin;

Zevalin™;

Zinecard® (Dexrazoxane);

Zoladex® (Goserelin);

Zoledronic acid;

Zolinza;

Zometa® (Zoledronic Acid for Inj).

Antibiotics

Antibiotics for use in the present invention include some of the newer antibiotics, penicillins, cephalosporins, monobactams, carbapenems, macrolides, aminoglycosides, quinolines (including fluoroquinolones), sulfonamides, tetracyclines and other antibiotics, among others.

The newer antibiotics include, for example daptomycin, gemifloxacin, telavancin, ceftaroline, and fidaxomicin, among others;

The penicillins, include for example amoxicillin, ampicillin, bacampicillin, carbenicillin, cloxacillin, dicloxacillin, flucloxacillin, mezlocillin, nafcillin, oxacillin, penicillin G, penicillin V, piperacillin, pivampicillin, pivmecillinam and ticarcillin, among others;

The cephalosporins, include for example cefacetrile (cephacetrile), cefadroxil (cefadroxyl), cefalexin (cephalexin), cefaloglycin (cephaloglycin), cefalonium (cephalonium), cefaloridine (cephaloradine), cefalotin (cephalothin), cefapirin (cephapirin), cefatrizine, cefazaflur, cefazedone, cefazolin (cephazolin), cefradine (cephradine), cefroxadine, ceftezole, cefaclor, cefamandole, cefmetazole, cefonicid, cefotetan, cefoxitin, cefprozil (cefproxil), cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime and ceftioxide, among others;

The monobactams, include for example, aztreonam, among others;

The carbapenems include for example, imipenem, imipenem/cilastatin, doripenem, meropenem and ertapenem, among others;

The macrolides, include for example, azithromycin, erythromycin, clarithromycin, dirithromycin, roxithromycin and telithromycin, among others;

The lincosamides, include for example, clindamycin and lincomycin, among others;

The streptogramins, include for example, pristinamycin and quinupristin/dalfopristin, among others;

The aminoglycosides, include for example, amikacin, gentamicin, kanamycin, neomycin, netilmicin, paromomycin, streptomycin and tobramycin, among others;

The quinolones, include for example, flumequine, nalidixic acid, oxolinic acid, piromidic acid, pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, parfloxacin, temafloxacin, tosufloxacin, besifloxacin, clinafloxacin, gemifloxacin, sitafloxacin, trovafloxacin and prulifloxacin, among others;

The sulfonamides, include for example, sulfamethizole, sulfamethoxazole, sulfisoxazole and trimethoprim-sulfamethoxazole (bactrim), among others,

The tetracyclines, include for example, demeclocycline, doxycycline, minocycline, oxytetracycline, tetracycline and the glycylcyclines, including tigecycline, among others,

Other antibiotics include for example, chloramphenicol, metronidazole, tinidazole, nitrofurantoin, the glycopeptides such as vancomycin and teicoplanin, the lipoglycopeptides such as telavancin, the oxazolidinones, such as linezolid. and cycloserine 2, the rifamycins, such as rifampin, rifabutin and rifapentine, the polypeptides, such as bacitracin and polymyxin B and the tuberactinomycins, including viomycin and capreomycin

Immunosuppression Agents

Zn (zinc salt hydrate(s), preferably zinc sulfate heptahydrate, either alone or in combination with another zinc salt hydrate, and optionally, at least one pharmaceutically acceptable water-soluble zinc salt non-hydrate) plus agents used as immunosuppressive agents following organ transplantation such as cyclosporine and its derivatives, azathioprine, 6-mercaptopurine, Prednisone, infliximab (Remicade), and tetracycline, among others.

Assorted Medications

Zn (zinc salt hydrate(s), preferably zinc sulfate heptahydrate, either alone or in combination with another zinc salt hydrate, and optionally, at least one pharmaceutically acceptable water-soluble zinc salt non-hydrate) given in combination with asthma related drugs: Theophylline and cortisteroids, including betamethasone, cortisone, dexamethasone, hydrocortisone, methylprednisolone, prednisolone and budenoise. Each of these agents can induce stomach lining erosion and increased acid secretion.

NSAIDS:

Over the counter NSAIDS and associated compounds cause GERD and GERD symptoms including ulcer disease:

| OTC Name | Generic Name |
|---|---|
| Actron ® | ketoprofen |
| Advil ® | ibuprofen |
| Aleve ® | naproxen sodium |
| Bayer ® | aspirin |
| Ecotrin ® | aspirin |
| Excedrin ® | aspirin, acetaminophen and caffeine |
| Motrin IB ® | ibuprofen |
| Nuprin ® | ibuprofen |
| Orudis KT ® | ketoprofen |

Blood Thinners

Blood thinners, including arixtra, clopidogrel, coumadin, dipyidamole (alone or with acetyl salicylic acid-asa), fragmin, lovenox, lovenox Hp, plavix, prasugrel and mixtures thereof.

Antidepressive Agents

Selective serotonin reuptake inhibitors (SSRIs): Citalopram (Celexa), Escitalopram (Lexapro), Fluoxetine (Prozac); Parozxetine (Paxel) and Sertraline (Zoloft.)

Zn (zinc salt hydrate(s), preferably zinc sulfate heptahydrate, either alone or in combination with another zinc salt hydrate, and optionally, at least one pharmaceutically acceptable water-soluble zinc salt non-hydrate) taken in combination with the following drugs could potentially increase bioavailability of molecule due to the fact zinc does not cause the effects on inhibiting Cytochromes P-450 2C9 & 3A4 as omeprazole or esoprazole.
Carbamazepine;
Cyclosporine;
Diazepam, other benzodiazepines;
Diltiazem, Nifedipine, Verapamil;
Erythromycin, Clarithromycin;
Lidocaine;
Lovastatin, other statins including Atorvastatin,
Phenytoin;
Quinidine;
Terfenadine

The term “proton pump inhibitor” is used throughout the specification to describe Proton pump inhibitors as drugs that help control the painful discomfort of heartburn and gastroesophageal reflux disease (GERD), and promote the healing of stomach and duodenal ulcers. Proton pump inhibitors are only available by prescription. They come as tablets, capsules, injections, or powders that are made into a suspension.

Proton inhibitors work by blocking the production of stomach acid. They inhibit a system in the stomach known as the proton pump, which is another name for the “hydrogen-potassium adenosine triphosphate enzyme” system. Proton pump inhibitors are rather versatile. They are used to heal stomach and duodenal ulcers, including stomach ulcers caused by taking nonsteroidal anti-inflammatory drugs. They are also used to relieve symptoms of oesophagitis (inflammation of the oesophagus or gullet) and severe gastroesophageal reflux (GERD), as discussed above.

Combined with certain antibiotics (such as amoxycillin and clarithromycin) or with zinc salts according to the present invention (zinc salt hydrate(s), preferably zinc sulfate heptahydrate, either alone or in combination with another zinc salt hydrate, and optionally, at least one pharmaceutically acceptable water-soluble zinc salt non-hydrate), proton pump inhibitors are effective for treating Helicobacter pylori infections (a bacterial infection of the stomach). The H. pylori bacterium is a chief suspect in the cause of recurring stomach ulcers. PPIs are also a first-choice treatment for the rare condition called Zollinger-Ellison syndrome, discussed above.

Proton Pump Inhibitors exhibit side effects, although they tend to be manageable, including diarrhea, feeling or being sick (nausea), constipation, flatulence, abdominal pain, headaches and more rarely, allergic reactions, itching, dizziness, swollen ankles, muscle and joint pain, blurred vision, depression and dry mouth, among others. Long-term use of proton pump inhibitors can result in stomach infections. Because proton pump inhibitors completely stop acid production—and stomach acid helps kill microbes such as bacteria in the stomach—using PPIs can lead to growth of potentially harmful microbes in the stomach.

Proton pump inhibitors exhibit significant, sometimes deleterious drug interactions, including reactions with phenytoin as an epilepsy agent and warfarin to prevent blood clots, to increase their effects, with ketoconazole and itraconazole to reduce their absorptivity, with diazepam (valium) to decrease its metabolism.

Proton pump inhibitors are usually taken for 1-2 months, but in some cases may be taken longer. Symptoms may return when a person stops taking a proton pump inhibitor. Proton pump inhibitors may cause internal bleeding, signs of which include vomiting blood, detecting a substance-like coffee grounds in your vomit, or pass black tarry stools, see your doctor immediately.

Common proton pump inhibitors include omeprazole (Prilosec), esomeprazole (Nexium), lansoprazole (Prevacid), pantoprazole (Protonix) and rabeprazole sodium (Aciphex). Many of these agents may readily benefit from the administration of a zinc hydrate salt, especially zinc sulfate heptahydrate because of their tendency to be inactivated by stomach acid. Immediate release forms of these active agents and zinc hydrate salt(s) may be particularly advantageous for enhancing the bioavailability of the proton pump inhibitor and for providing compositions which combine an immediate pH raising effect from the zinc salt in combination with longer-term relief from the proton pump inhibitor. Sustained and/or controlled release forms of these active agents and zinc hydrate salt(s) may also be useful for providing an immediate and longer-term therapy.

The present invention relates to a method for providing fast action with optional long duration effect in reducing gastric acid secretion, raising the pH of the stomach during a resting phase, decreasing the duration of stomach acid release during a secretagogue phase and for treating conditions including gastroesophageal reflux disease (GERD), non-erosive reflux disease (NERD), Zollinger-Ellison syndrome (ZE disease), ulcer disease, and gastric cancer where the reduction in gastric acid secretion is beneficial, as well as preventing or reducing the likelihood of ulcer disease by reducing gastric acid section. In addition, the present methods are useful for treating patients who are non-responsive to proton pump inhibitors (PPI) and as an alternative to traditional therapies or conditions which are caused by rapid and complete inhibition of secretagogue induced acid secretion.

The method comprises administering an effective amount of zinc salt hydrate(s), preferably zinc sulfate heptahydrate, either alone or in combination with another zinc salt hydrate, and optionally, at least one pharmaceutically acceptable water-soluble zinc salt non-hydrate, to alleviate or treat the condition or disease state. The methods may involve the administration of zinc salt hydrate(s), preferably zinc sulfate heptahydrate, either alone or in combination with another zinc salt hydrate, and optionally, at least one pharmaceutically acceptable water-soluble zinc salt non-hydrate, and optionally other agents as disclosed herein a single time, or preferably for longer duration, usually about 2-3 days to about 2-3 months, with varying intervals in between, depending upon the prognosis and outcome of the treatment.

Zinc salt hydrate(s), preferably zinc sulfate heptahydrate, either alone or in combination with another zinc salt hydrate, and optionally, at least one pharmaceutically acceptable water-soluble zinc salt non-hydrate, may be administered alone or in further combination with other compounds, compositions or therapies, depending upon the condition or disease state to be treated, including an effective amount of a proton pump inhibitor or other agent as otherwise described herein which may be used to treat H. pylori infections. These agents include proton pump inhibitors such as esomeprazole, lansoprazole, omeprazole, pantoprazole or rabeprazole, antacids, including bicarbonate (sodium, potassium, calcium, magnesium, etc.), carbonates (sodium, potassium, magnesium and calcium), hydroxide (aluminum and magnesium), which in certain embodiments are excluded from the composition and bismuth subsalicylate, H2 blockers such as cimetidine, famotidine, nizatidine or ranitidine, anti-H. pylori agents, such amoxicillin, clarithromycin (biaxin), metronidazole (flagyl) or tetracycline, cytoprotective agents such as bismuth subsalicylate or sucralfate, a combination agent such as Helidac or Prevpac, and mixtures thereof. In certain embodiments, an effective amount of copper is administered to the patient or subject at least one hour after the administration of the zinc salt-containing composition, often at least 2, 3, 4, 5, 6, 7, 8 or more hours after the administration of the zinc-containing composition.

In a preferred aspect of the invention, the administration of zinc salt hydrate(s), preferably zinc sulfate heptahydrate alone or in combination with an additional zinc salt hydrate further includes at least one additional (water-soluble) zinc salt which is characterized as being soluble and absorbable (through the gastrointestinal mucosa) at both low pH (i.e., a pH of about 1-2, which occurs in an acidic condition during secratogogue in the stomach) and higher pH (i.e., a pH of about 4-5 or slightly above after acid secretion in the stomach is inhibited or even higher—i.e., a pH of about 5.5-6.0 in the duodenum to about 6.5-7.5 in the jejunum and ileum—the pH is slightly higher in the ileum than in the jejunum). By providing for compositions which include additional zinc salts which are both water-soluble and absorbable throughout the gastrointestinal mucosa (i.e. in the stomach and through the various sections of the small intestine), the bioavailability of the zinc salt will be maximized as will favorable therapy of the conditions or disease states to be treated as a consequence of an immediate effect from the zinc sulfate hydrate(s), preferably zinc sulfate heptahydrate and a longer duration effect from the additional zinc salt. In this aspect, zinc salt hydrate(s), especially including zinc sulfate heptahydrate alone or in combination with an additional zinc salt hydrate may be combined with effective amounts of a zinc salt preferably selected from the group consisting of zinc acetate, zinc arginate, zinc butyrate, zinc chloride, zinc citrate, zinc formate, zinc fumarate, zinc gluconate, zinc glutarate, zinc glycerate, zinc glycolate, zinc histidinate, zinc lactate, zinc malate, zinc maleate, zinc picolinate, zinc propionate, zinc salicylate, zinc succinate, zinc sulfate, zinc undecylenate, zinc salt of 1,6 fluctose diphosphate and mixtures thereof, more preferably, zinc chloride, zinc acetate, zinc gluconate, zinc ascorbate, zinc succinate and a zinc amino acid chelate (as mono- or bis-amino acid chelate) is preferred, although numerous other zinc acid compounds may be combined to produce favorable results.

In addition to zinc salt hydrate(s), especially zinc sulfate heptahydrate, a number of additional zinc salts may be combined with zinc salt hydrate (s), including zinc sulfate heptahydrate. Preferred additional zinc salts include those salts in which the anionic counterion in protonated form has a pKa of at least about 4 to about 5.5 or higher. Mixtures of zinc salts wherein all of the zinc salts are soluble within a range of pH from 1-2 to about 7.5 are preferred. Zinc chloride, zinc acetate, zinc citrate, zinc gluconate, zinc glycolate, zinc lactate, zinc picolinate, zinc succinate and zinc ascorbate alone or in combination with another zinc salt may often be useful in the present invention. Zinc chelates, especially zinc amino acid chelates (mono- or bis-amino acid chelates) may also be used, the zinc amino acid chelate being selected from the group consisting of zinc chelates (mono- or bis-chelates) of L-cysteine, L-cystine, L-N-acetylcysteine, L-histidine, D-histidine, L-taurine, L-glycinate, L-aspartate, L-methionine, and mixtures thereof.

Note that the following zinc salts have solubilities which tend to be reduced at pH values above about 7.0, so approaches to formulation should accommodate such information where absorptivity from the small intestine is featured (duodenum, jejunum and ileum), especially at the distil end (jejunum, ileum) where the pH of the small intestine may rise to between 7-8.0. Note that the use of such salts may favorably influence release characteristics of formulations and provide a means of delivering therapeutic agents, especially those which are administered in combination therapy according to the present invention. Zinc salts that become insoluble above a pH of 7 include zinc acetate, zinc chloride, zinc bromide, zinc fluoride, zinc iodide, zinc sulfate, zinc citrate, zinc lactate, zinc nitrate, zinc propionate, zinc salicylate, zinc tartrate, zinc valerate, zinc gluconate, zinc selenate, zinc benzoate, zinc formate, zinc glycerophosphate, zinc picrate, zinc butyrate, and the like, and combinations thereof. These may be used appropriately in combination with zinc sulfate heptahydrate to enhance the therapeutic effect.

Preferred zinc salts that may be used in the present invention in certain embodiments in combination with zinc salt hydrate(s), especially including zinc sulfate heptahydrate, include zinc chloride (where pKa of the counterion is not important because of its interaction with chloride channels) and organic acids including zinc acetate (pka 4.75), zinc gluconate, zinc succinate, zinc tartrate, zinc malate, zinc maleate, zinc zinc ascorbate (pka of 4.2 and 11.6). Other zinc salts of organic acids may also be preferred, depending on the context of use. In addition, zinc glycolate and zinc lactate may also be used. Other preferred salts include, for example, zinc acetate, zinc arginate, zinc butyrate, zinc chloride, zinc citrate, zinc formate, zinc fumarate, zinc gluconate, zinc glutarate, zinc glycerate, zinc glycolate, zinc histidinate, zinc lactate, zinc malate, zinc maleate, zinc picolinate, zinc propionate, zinc salicylate, zinc succinate, zinc sulfate, zinc undecylenate, zinc salt of 1,6 fluctose diphosphate and mixtures thereof, with zinc chloride, zinc acetate, zinc gluconate, zinc lactate and/or zinc picolinate being preferred. In certain aspects of the invention, it is preferred that when a another zinc salt is used in combination with a zinc salt hydrate, especially zinc sulfate heptahydrate, at least one of the additional zinc salts is effective at a higher pH in the stomach (pH of 4.0-5.0 or higher), or alternatively, the additional zinc salt may be preferentially absorbed in the small intestine (a zinc mono- or bis-amino acid chelate or other chelate). Of course, in certain embodiments, a combination of zinc salts exhibiting the above characteristics may be combined with zinc sulfate heptahydrate to maximize favorable therapeutic effect.

While not being limited by way of theory, it is believed that a zinc salt hydrate, preferably zinc sulfate heptahydrate and optionally (preferably) one or more of the zinc salts as otherwise disclosed herein (especially zinc chloride, zinc acetate, zinc gluconate, zinc lactate and/or zinc picolinate) which are effective at a higher pH, will maximize delivery of zinc to the stomach mucosa to obtain a favorable effect, at first by being dissolved in acid gastric juice in the stomach where an initial inhibition of acid occurs and the pH rises, and subsequently, through absorption of zinc (from an additional zinc salt) at a higher pH in the stomach or in the small intestine where blood levels of zinc will increase to therapeutic levels. The absorption and effect of a zinc salt at higher pH levels in the stomach or at the higher pH of the small intestine (5.5-7.5 or higher) is advantageous in certain embodiments because this delayed absorption of zinc will reduce gastric acid secretion at a later time (than an initial effect at a low pH) over an extended period of time. Compositions according to the present invention may be administered a single time, but usually are administered preferably once or twice daily orally for a period ranging from about 2-3 days to several months or longer.

Compositions according to the present invention also relate to sustained or extended release formulations which comprise a first active component in immediate release form which contains a zinc salt hydrate(s), preferably, zinc sulfate heptahydrate alone or in combination with another zinc salt hydrate and optionally, at least one additional active agent which allows or facilitates fast dissolution in the gastric juices at low pH so that a rapid inhibition of acid secretion is effected (with concombinant increase in pH to a level of about 3-4.0 or higher) and a second component which releases zinc salt and/or another active agent at the higher pH level in the stomach or more preferably, further in the small intestine on a sustained release basis in order to maintain an effective level of zinc in the blood stream to inhibit gastric acid secretion in the stomach for extended periods. The first fast-acting component may be readily formulated using zinc salt hydrate(s), especially including zinc sulfate heptahydrate alone or in combination with another zinc salt hydrate and optionally, at least one additional agent (which may include an additional zinc salt or another therapeutic agent), which dissolves in gastric juice at low pH or higher pH (after the effect of the zinc salt hydrate, especially zinc sulfate heptahydrate raising pH becomes manifest) using standard excipients such as lactose, confectioner's sugar in powered form, various stearate salts, etc, which dissolves rapidly in the stomach and a second sustained or extended release formulation which makes use of any number of polymeric binders, matrices (polymeric and/or erodible), granules, or enteric coatings to allow release of a second zinc salt and/or a therapeutic agent on an extended or sustained release basis in the small intestine. Many of these techniques are well known in the art. Exemplary patents such as U.S. Pat. No. 4,863,741 to Becker, U.S. Pat. No. 4,938,967 to Newton, et al., U.S. Pat. No. 4,940,556 to MacFarlane, et al., and U.S. Pat. No. 5,202,128 to Morella, et al., among numerous others, may be useful for providing teachings, all well known in the art, for formulating fast release/sustained or extended release formulations useful in the present invention.

The above formulations may be useful for providing enhanced bioavailability of one or more zinc salts other than zinc salt hydrate(s), especially zinc sulfate heptahydrate and optionally, other agents as described herein which may be useful in treating or reducing the likelihood of one or more of gastric ulcers, GERD, NERD, Zollinger-Ellison syndrome, gastric cancer and reducing/inhibiting the secretion of acid in the stomach, among numerous others, and raising the pH of the stomach to about 4.0 to about 5.0 or higher, as otherwise disclosed in the present invention. It is noted that in inhibiting acid secretion in the stomach, the use of zinc sulfate heptahydrate or other zinc salt hydrate of about 0.3 μM to about 300 μM (often at least about 1 μM within this range), more often about 3 μM to about 30 μM of zinc sulfate heptahydrate, produces almost complete inhibition. It may be shown that inhibition occurs within about 10-15 minutes to 1 about hour in the presence of secretagogue. The zinc salts may be administered orally, alone or in combination with optional therapeutic agents as otherwise described herein, especially including PPI drugs. Oral dosage forms which avoid aqueous solutions, e.g., powders, capsules, tablets and the like, may be preferred over aqueous solutions, in order to provide storage stability and maintain activity over substantial periods of storage (e.g. for at least one month up to several years, often at least 6 months or a year to several years).

As presented in the present invention, the use of zinc salt hydrate(s), especially zinc sulfate hepathydrate alone, or in combination with another zinc salt hydrate, optionally, in combination with at least one additional zinc salt as otherwise described herein may be used. Additional preferred zinc salts include zinc chloride, zinc acetate, zinc gluconate, zinc ascorbate, zinc lactate, zinc picolinate, zinc citrate, zinc succinate, and zinc amino acid chelates (mono- and bis-amino acid chelates). These zinc salts and combinations may be used alone or in combination with additional agents such as a proton pump inhibitor (esomeprazole, lansoprazole, omeprazole, pantoprazole or rabeprazole), an H2 blocker (cimetidine, famotidine, nizatidine or ranitidine), an anti-H. pylori agent (amoxicillin, clarithromycin, metronidazole or tetracycline), a cytoprotective agent such as bismuth subsalicylate or sucralfate, or a combination agent such as Helidac or Prevpac, as otherwise described herein. Additional therapeutic agents combined with zinc sulfate heptahydrate alone or in combination with additional zinc salts in immediate release form may take advantage of a raised pH profile in the stomach to enhance bioavailability or other attributes of the therapeutic agent in a raised pH of gastric juices, as otherwise described

In certain embodiments of the invention, pharmaceutical compositions comprise an effective amount of a zinc sulfate hydrate, especially zinc sulfate heptahydate alone, or in certain embodiments in combination with another zinc salt hydrate and optionally, at least one other zinc salt and/or an effective amount of at least one additional agent such as a traditional proton pump inhibitor such as such as esomeprazole, lansoprazole, omeprazole, pantoprazole or rabeprazole, an antacid, including bicarbonate (sodium, potassium, calcium, magnesium, etc.), carbonates (sodium, potassium, magnesium and calcium), hydroxide (aluminum and magnesium) which may be less preferred in certain embodiments where acid or pH rebound as a side effect occurs, bismuth subsalicylate, an H2 blocker such as cimetidine, famotidine, nizatidine or ranitidine, an anti-H. pylori agent, such amoxicillin, clarithromycin (biaxin), metronidazole (flagyl) or tetracycline, a cytoprotective agent such as bismuth subsalicylate or sucralfate, a combination agent such as Helidac or Prevpac, and mixtures thereof, optionally in combination with a pharmaceutically acceptable carrier, additive or excipient. In certain embodiments, where copper deficiency becomes an issue because of the administration of a zinc salt, an effective amount of copper may be administered at least one hour after zinc salt administration (in whatever form) and often 2, 3, 4, 5, 6, 7, 8 or more hours after zinc salt administration to increase potential depleted levels of copper in the patient or subject. In other embodiments, as discussed, a therapeutic agent in immediate release form with zinc salt hydrate, including zinc sulfate heptahydrate alone or in combination another zinc salt hydrate and optionally, another zinc salt may also be used to enhance the bioavailability (including reducing the acid instability) of the therapeutic agent or other attribute as otherwise described herein.

Pharmaceutical formulations according to the present invention include those suitable for oral (including oral and/or nasal intubation directly into the stomach and gastric juices), rectal, nasal, topical (including buccal and sub-lingual), vaginal or parenteral (including intramuscular, sub-cutaneous and intravenous) administration. Oral compositions or parenteral compositions (especially those for IV administration) may be preferred. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. Oral compositions which are based upon aqueous solutions may be best prepared just prior to administration in order to promote the stability of the zinc sulfate heptahydrate. In this embodiment, a powder or other composition which is not in aqueous solution form is placed in aqueous solution, optionally with one or more additional agents, just prior to oral administration to provide a particularly active formulation. Alternatively, when compositions are provided in aqueous solution, an effective amount of a stabilizer such as glycerine, ethanol, propylene glycol, PEG (especially PEG 200-PEG 1000, preferably PEG 400) and mixtures thereof may be included in the aqueous solution to promote the stability of the active species of zinc which occurs when zinc sulfate heptahydrate or other zinc salt hydrate is placed in aqueous solution. The amount of stabilizer which is used in these solutions may be as much as 10% or more of the total solvent used in the solution. In certain embodiments, zinc salt hydrate solution compositions are provided which are aqueous free to promote storage stability of the composition. In these aqueous free compositions, the amount of stabilizer which is included in zinc salt hydrate solutions, especially zinc sulfate heptahydrate solutions may be as much as 100% of the total solvent used to provide the solution. These solutions are stored prior art use and used directly or formulated for oral delivery by added water just prior to oral administration.

As discussed hereinabove, when desired, the present formulations may be adapted to provide sustained release characteristics of the active ingredient(s) in the composition using standard methods well-known in the art. A composition which provides an effective amount of initial dose of zinc sulfate heptahydrate or other zinc hydrate in the gastric juice at low pH followed by extended release effects of zinc over a longer duration may be used as well. Oral gel capsules which utilize a non-aqueous solvent such as ethanol and/or glycerine among others, may be used to enhance stability of the compositions over longer periods of time to maintain the zinc salt hydrate, especially the zinc sulfate heptahydrate in its active form (as the heptahydrate). Other oral compositions in capsule and/or tablet form may also be used.

In the case of combination pharmaceutical compositions, i.e., a composition which comprises zinc salt hydrate(s), preferably zinc sulfate hydrate alone or in combination with another zinc salt hydrate and optionally, at least one additional water soluble salt in combination with a therapeutic agent (i.e., other than the zinc salt(s), compositions may be formulated in admixture or they may be compartmentalized in the dosage form. Pharmaceutical formulations may be formulated in admixture by mixing the actives together along with the pharmaceutically acceptable carriers, additives and/or excipients in powder or liquid form and then using them directly or presenting the mixture in tablet or capsule form. The compositions may be immediate release, sustained or controlled release or intermediate sustained or controlled release or any combination thereof in a single dosage form, depending upon the results desired. Formulations may also be presented which compartmentalize the zinc salt hydrate, especially zinc sulfate heptahydrate, the optional additional zinc salt and the therapeutic agent into more than one portion of a tablet or capsule to take advantage of differential solubilities in order to enhance the bioavailability of the zinc salt hydrate, preferably zinc sulfate heptahydrate, the additional zinc salt and/or the additional therapeutic agents, using methods which are readily available in the art to those of ordinary skill.

In the pharmaceutical aspect according to the present invention, the compound(s) according to the present invention is formulated preferably in admixture with a pharmaceutically acceptable carrier. In general, it is preferable to administer the pharmaceutical composition orally (including through oral or nasal intubation), but certain formulations may be preferably administered parenterally and in particular, in intravenous or intramuscular dosage form, as well as via other parenteral routes, such as transdermal, buccal, subcutaneous, suppository or other route, including via inhalation intranasally. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

In certain preferred embodiments, the present compositions are preferably readily water soluble and mixtures of zinc sulfate(s), especially zinc sulfate heptahydrate alone or in combination with another zinc salt hydrate and optionally additional water-soluble zincs may be used to effect an immediate release/sustained release pharmaceutical profile. This may maximize immediate effect and longer duration effect by simply choose the type of salt and adjusting the ratio of the zinc salt mixture accordingly. Of course, excipients can be chosen to affect the delivery and bioequivalence of the zinc salts used. It is well within the routineer's skill to modify the route of administration and dosage regimen of a particular compound in order to manage the pharmacokinetics of the present compounds for maximum beneficial effect to the patient.

Formulations containing the compounds of the invention may take the form of solid, semi-solid, lyophilized powder, or liquid dosage forms, such as, for example, tablets, capsules (including gel capsules), powders, sustained-release formulations, solutions (preferably, stabilized), suspensions, emulsions, suppositories, creams, ointments, lotions, aerosols or the like, preferably in unit dosage forms suitable for simple administration of precise dosages.

The compositions typically include a conventional pharmaceutical carrier or excipient and may additionally include other medicinal agents, carriers, and the like. Preferably, the composition will be about 0.05% to about 75-80% by weight of a zinc salt compound or compounds according to the invention, with the remainder consisting of suitable pharmaceutical additives, carriers and/or excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20%), and optional pharmaceutical additives, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

The present invention also contemplates a route of administration other than an oral route. An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Methods for preparing such dosage forms are known or will be apparent to those skilled in the art; for example, see “Remington's Pharmaceutical Sciences” (17th Ed., Mack Pub. Co., 1985). The person of ordinary skill will take advantage of favorable pharmacokinetic parameters of the pro-drug forms of the present invention, where applicable, in delivering the present compounds to a patient suffering from a viral infection to maximize the intended effect of the compound.

The pharmaceutical compositions according to the invention may also contain other active ingredients such as proton pump inhibitors, antacids (acid neutralizing agents, which are avoided in certain embodiments because of acid round side effects), H₂ blockers, antimicrobial agents, cytoprotective agents or combination agents, and mixtures thereof. In addition, compounds according to the present invention may also contain anti-cancer agents (to treat gastric cancer), and antibiotics, among numerous others as described herein. Effective amounts or concentrations of each of the active compounds are to be included within the pharmaceutical compositions according to the present invention.

The individual components of such combinations may be administered either sequentially or simultaneously in separate or combined pharmaceutical formulations.

When one or more of the compounds according to the present invention is used in combination with a second therapeutic active agent the dose of each compound may be either the same as or differ from that when the compound is used alone. Appropriate doses will be readily appreciated by those skilled in the art.

The following examples are used to describe the present invention. It is understood that they are merely exemplary and are understood not to limit the breadth of the invention in any way.

### Examples

The stomach produces acid to help break down food, making it easier to digest. In some cases, stomach acid can actually irritate the lining of the stomach and the duodenum (top end of the small intestine). Sometimes the acid “refluxes” upwards and irritates the lining of the esophagus. Irritation of the lining of the stomach or the esophagus causes acid indigestion (heartburn) and sometimes causes ulcers or bleeding.

We show in this particular application that zinc sulfate heptahydate has an unexpectedly potent inhibitory effect on gastric acid secretion at the cellular level by abolishing the activity of the gastric H⁺,K⁺-ATPase in rat and human gastric glands.

The following experiments were conducted to evaluate the effects of Zinc Sulfate Heptahydrate (ZnSO₄7H₂O) on gastric acid secretion. These studies used a combination of single hand dissected glands from rats and humans. In addition we also performed studies on whole stomachs and the effects of acute single dose exposure to (ZnSO₄7H₂O) in the presence or absence of a known stimulator of acid secretion. Finally studies were also carried out on chronic exposure to ZnSO₄7H₂O over a 7 day period where we added the ZnSO₄7H₂O to the drinking water.

Protocol 1 Dose Dependence of ZnSO47H2O as an Inhibitor of Secretory Induced Acid Secretion.

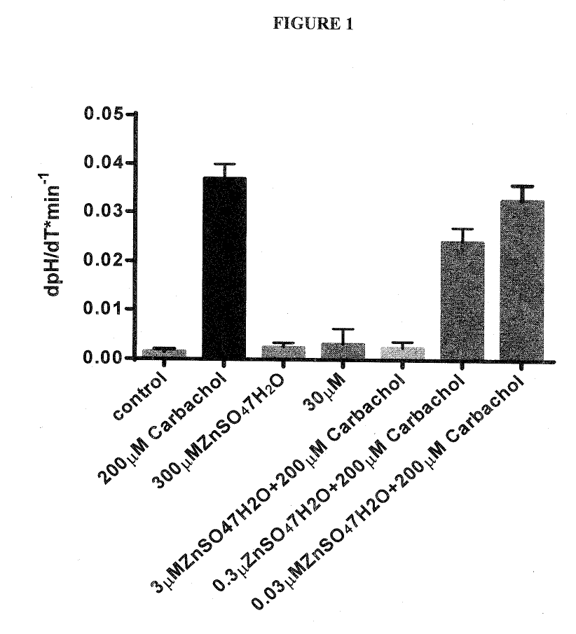

In this series of studies we used glands from rats that have been fasted for 18 hours with free access to water to reduce basal acid secretion to zero. Following isolation the glands were placed in a perfusion chamber that was maintained at 37° C. Prior to exposure to the Zn solution the glands were loaded with the Fluorescent reagent BCECF to monitor intracellular pH, this is based on technology we have used in the lab. In these studies we stimulated acid secretion by exposing the individual glands to 200 μM Carbachol a well characterized activator of acid secretion. In parallel to these studies (Control glands) we exposed glands from the same animal to a solution that contained ZnSO4.7H₂O. In the initial studies we started at a concentration of 300 μM a dose that was from our previous studies known to cause a dramatic reduction in acid secretion in the secretagogue stimulated gland. In subsequent studies we then reduced the concentration of ZnSO4.7H₂O by an order of magnitude (330-30 μM) and repeated the study. For each dose used a minimum of 5 animals was used. Based on the initial results we continued to reduce by an order of magnitude (30-3-.3-.03) until we reached a concentration that had minimal effects on acid secretion. We reduced to a concentration of 0.03 μM. The results of these studies are presented in the summary graph in FIG. 1.

FIG. 1 shows modulation of proton extrusion rates (acid secretion) following exposure to the secretagogue Carbachol (200 μM) in the presence of various concentrations of ZnSO4.7H₂O in the rat. Please note that all doses from 300 μM-0.3 μM gave a highly significant inhibition of proton extrusion (p<0.0001). At a dose of 0.03 μM there was still a modest yet significant inhibition of acid secretion (p<0.03) All data are the means from 5 individual rats.

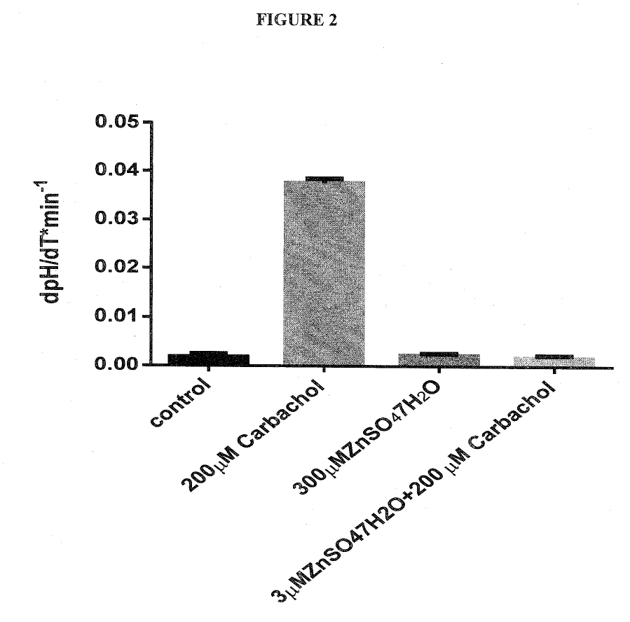

FIG. 2 shows modulation of proton extrusion rates (acid secretion) following exposure to the secretagogue Carbachol (200 μM) in the presence of various concentrations of ZnSO4.7H₂O in isolated human glands. Note that doses of 300 μM and 3 μM were used. Both doses in isolated human glands gave a highly significant inhibition of proton extrusion (p<0.0001). All data are the means from 5 separate human samples.

The results from the studies in Protocol 1 demonstrate that ZnSO4.7H₂O is a potent inhibitor of secretagogue induced acid secretion in isolated gastric glands both in rats and in humans. The data suggest that we can effectively reduce the dose to 1/100 of the dose that we have used for other Zinc salts and still have a complete inhibition of acid secretion.

Protocol 2 Effect of ZnSO47H2O on Intragastric pH in a Rat Model

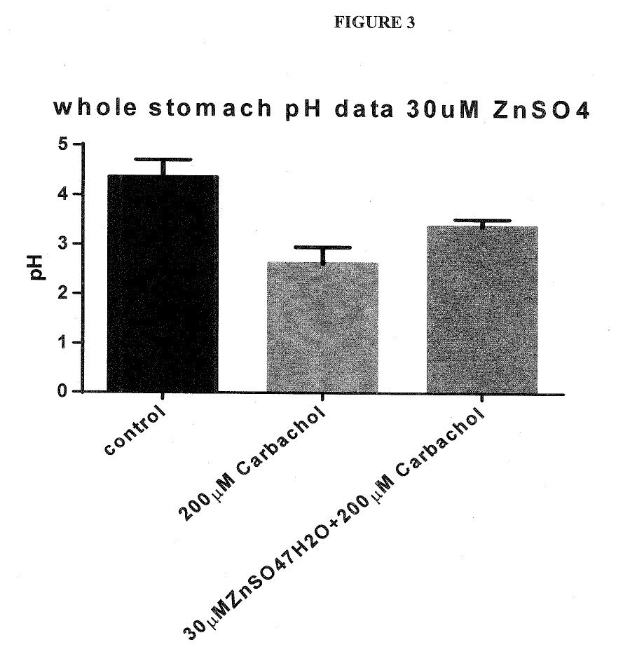

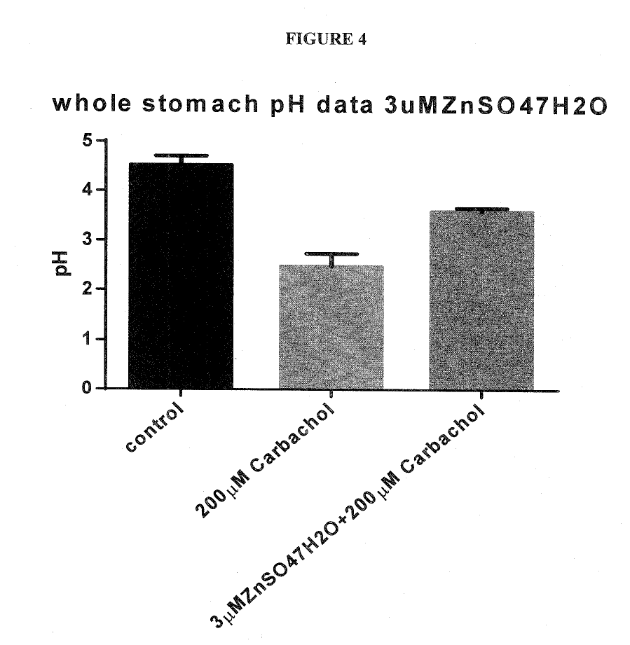

In this series of studies we chose the doses of 30 μM and 3 μM of ZnSO4.7H₂O two of the most effective cellular concentrations of ZnSO4.7H₂O; (obtained from the results of Protocol 1 above) to load stomachs from 18 hour fasted rats ex vivo. We took age and sex matched animals for these studies. The stomach was excised and perfused with 0.5 ml of nonbuffered saline solution in vitro with a solution of either 200 μM Carbachol or ZnSO4.7H₂O+200 μM Carbachol following a 15 minute perfusion time we collected the effluent from the stomach and measure intragastric pH in this fluid following a protocol that we have used in the past. As shown in FIG. 3 and FIG. 4 both 30 μM and 3 μM of ZnSO4.7H₂O are effective in acute conditions to raise intragastric pH in the presence of a known secretagogue.

Protocol 3 Prolonged Exposure of ZnSO47H2O on Gastric Acid Secretion and Morphology

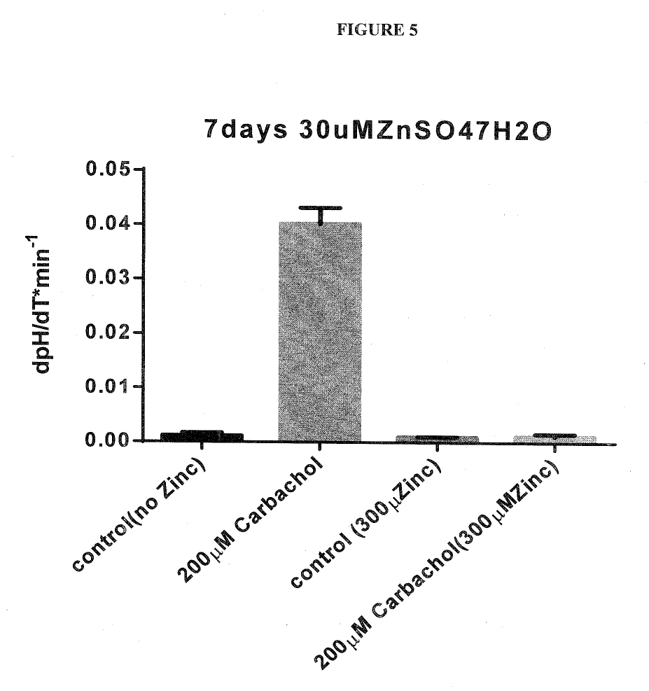

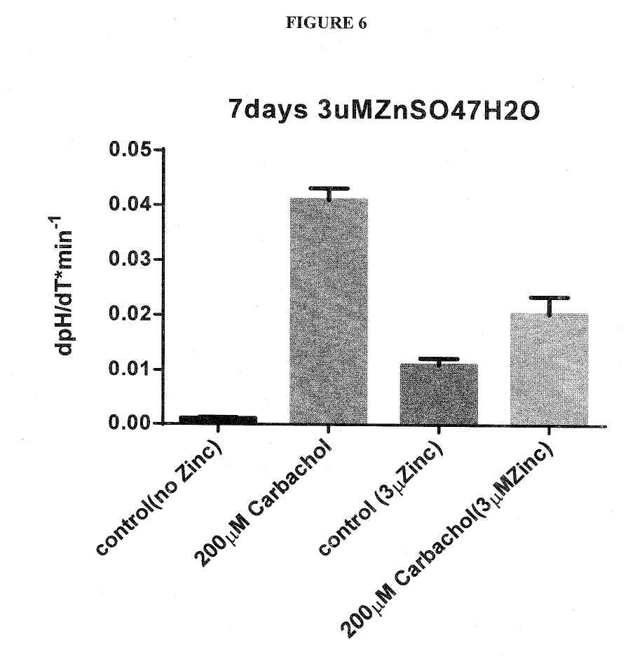

In this series of studies Rats were given free access to water and normal rat chow for a period of 5 days. In one group 30 μM ZnSO4.7H₂O was added to the drinking water so that the animals are getting a constant exposure of ZnSO4.7H₂O. At the end of the 5 day period the animals were sacrificed and the stomach removed. We examined how these glands performed to addition of a secretagogue as previously defined in Protocol 1. In the second group we repeated the study giving 3 μM ZnSO4.7H₂O for the 5 day period while allowing normal access to food and water. As shown in FIG. 5 and FIG. 6 we show that there appears to be a memory effect so that acid secretion is suppressed at the cellular level.

### Summary

In this series of sponsored research studies we set out to ask the question of whether ZnSO4.7H₂O was a potential inhibitor of acid secretion in rats and humans. We have in this series been able to demonstrate that ZnSO4.7H₂O can be used at a dosage range from 300 to 0.3 μM in both the rat and the human. Furthermore we were able to demonstrate that we can give a solution of either 30 or 3 μM ZnSO4.7H₂O acutely to whole stomachs and raise the pH of the stomach by 1 pH unit and sustain this in the presence of a potent secretagogue Carbachol. Finally we were able to show that chronic exposure (7 days) to either a 30 or 3 μM ZnSO4.7H₂O solution has a “memory effect” so that acid secretion at the cellular level remain suppressed when glands are excised and exposed acutely to a secretagogue (200 μM Carbachol).

We conclude that ZnSO4.7H₂O is a potent inhibitor of acid secretion both acutely and chronically at a dose much lower than other zinc salts we have previously tested.

### REFERENCES

1. Hersey S J, Sachs G. Gastric acid secretion. Physiol Rev 1995; 75:155-189.
2. Sachs G, Prinz C, Loo D, Bamberg K, Besancon M, Shin J M. Gastric acid secretion: activation and inhibition. Yale J Biol Med 1994; 67:81-95.
3. Sachs G. Physiology of the parietal cell and therapeutic implications. Pharmacotherapy 2003; 23:68S-73S.
4. Horie S, Yano S, Watanabe K. Effects of drugs acting on Cl(−)−. Eur J Pharmacol 1992; 229:15-19.
5. Helander H F, Keeling D J. Cell biology of gastric acid secretion. Baillieres Clin Gastroenterol 1993; 7:1-21.
6. Soumarmon A, Lewin M J. Gastric (H+,K+)-ATPase. Biochimie 1986; 68:1287-1291.
7. Wolfe M M, Welage L S, Sachs G. Proton pump inhibitors and gastric acid secretion. Am J Gastroenterol 2001; 96:3467-3468.
8. Aihara T, Nakamura E, Amagase K, Tomita K, Fujishita T, Furutani K, Okabe S. Pharmacological control of gastric acid secretion for the treatment of acid-related peptic disease: past, present, and future. Pharmacol Ther 2003; 98:109-127.
9. Gardner J D, Sloan S, Miner P B, Robinson M. Meal-stimulated gastric acid secretion and integrated gastric acidity in gastro-oesophageal reflux disease. Aliment Pharmacol Ther 2003; 17:945-953.
10. Williams J L. Gastroesophageal reflux disease: clinical manifestations. Gastroenterol Nurs 2003; 26:195-200.
11. Lehmann F, Hildebrand P, Beglinger C. New molecular targets for treatment of peptic ulcer disease. Drugs 2003; 63:1785-1797.
12. Brzozowski T, Konturek P C, Konturek S J, Drozdowicz D, Kwiecien S, Pajdo R, Bielanski W, Hahn E G. Role of gastric acid secretion in progression of acute gastric erosions induced by ischemia-reperfusion into gastric ulcers. Eur J Pharmacol 2000; 398:147-158.
13. Franzin G, Manfrini C, Musola R, Rodella S, Fratton A. Chronic erosions of the stomach—a clinical, endoscopic and histological evaluation. Endoscopy 1984; 16:1-5.
14. Raugstad T S, Svanes K, Ulven A, Moister A. Interaction between acute gastric ulcer and epinephrine-induced mucosal erosions in the rat: the significance of gastric acid secretion. Digestion 1979; 19:70-72.
15. Houghton J, Stoicov C, Nomura S, Rogers A B, Carlson J, Li H, Cai X, Fox J G, Goldenring J R, Wang T C. Gastric cancer originating from bone marrow-derived cells. Science 2004; 306:1568-1571.
16. Bell N J, Hunt R H. Progress with proton pump inhibition. Yale J Biol Med 1992; 65:649-657.
17. Garnett W R. Lansoprazole: a proton pump inhibitor. Ann Pharmacother 1996; 30:1425-1436.
18. Robinson M. Drugs, bugs, and esophageal pH profiles. Yale J Biol Med 1999; 72:169-172.
19. Tutuian R, Katz P O, Castell D O. Nocturnal acid breakthrough: pH, drugs and bugs. Eur J Gastroenterol Hepatol 2004; 16:441-443.
20. Adachi K, Komazawa Y, Fujishiro H, Mihara T, Ono M, Yuki M, Kawamura A, Karim Rumi M A, Amano Y, Kinoshita Y. Nocturnal gastric acid breakthrough during the administration of rabeprazole and ranitidine in Helicobacter pylori-negative subjects: effects of different regimens. J Gastroenterol 2003; 38:830-835.
21. Kleinman L, McIntosh E, Ryan M, Schmier J, Crawley J, Locke G R, III, De L G. Willingness to pay for complete symptom relief of gastroesophageal reflux disease. Arch Intern Med 2002; 162:1361-1366.
22. Carlsson R, Galmiche J P, Dent J, Lundell L, Frison L. Prognostic factors influencing relapse of oesophagitis during maintenance therapy with antisecretory drugs: a meta-analysis of long-term omeprazole trials. Aliment Pharmacol Ther 1997; 11:473-482.
23. Katz P O, Hatlebakk J G, Castell D O. Gastric acidity and acid breakthrough with twice-daily omeprazole or lansoprazole. Aliment Pharmacol Ther 2000; 14:709-714.
24. Tytgat G N. Shortcomings of the first-generation proton pump inhibitors. Eur J Gastroenterol Hepatol 2001; 13 Suppl 1:S29-S33.
25. Gedda K, Scott D, Besancon M, Lorentzon P, Sachs G. Turnover of the gastric H+,K(+)-adenosine triphosphatase alpha subunit and its effect on inhibition of rat gastric acid secretion. Gastroenterology 1995; 109:1134-1141.
26. Diamond I, Hurley L S. Histopathology of zinc-deficient fetal rats. J Nutr 1970; 100:325-329.
27. Elmes M E, Jones J G. Ultrastructural studies on Paneth cell apoptosis in zinc deficient rats. Cell Tissue Res 1980; 208:57-63.
28. Fong L Y, Lee J S, Chan W C, Newberne P M. Zinc deficiency and the development of esophageal and forestomach tumors in Sprague-Dawley rats fed precursors of N-nitroso-N-benzylmethylamine. J Natl Cancer Inst 1984; 72:419-425.
29. Ng W L, Fong L Y, Ma L, Newberne P M. Dietary zinc deficiency and tumorigenesis: a scanning electron microscope study. J Electron Microsc (Tokyo) 1984; 33:344-348.
30. Sunderman F W, Jr. The influence of zinc on apoptosis. Ann Clin Lab Sci 1995; 25:134-142.
31. Cho C H, Fong L Y, Ma P C, Ogle C W. Zinc deficiency: its role in gastric secretion and stress-induced gastric ulceration in rats. Pharmacol Biochem Behav 1987; 26:293-297.
32. Cho C H, Fong L Y, Wong S H, Ogle C W. Zinc deficiency worsens ethanol-induced gastric ulcers in rats. Drug Nutr Interact 1988; 5:289-295.
33. Frommer D J. The healing of gastric ulcers by zinc sulphate. Med J Aust 1975; 2:793-796.
34. Watanabe T, Arakawa T, Fukuda T, Higuchi K, Kobayashi K. Zinc deficiency delays gastric ulcer healing in rats. Dig Dis Sci 1995; 40:1340-1344.
35. Naess K. [Zinc in the treatment of stomach ulcer]. Tidsskr Nor Laegeforen 1976; 96:1334.
36. Kirchhoff P, Wagner C A, Gaetzschmann F, Radebold K, Geibel J P. Demonstration of a functional apical sodium hydrogen exchanger in isolated rat gastric glands. Am J Physiol Gastrointest Liver Physiol 2003; 285:G1242-G1248.
37. McDaniel N, Lytle C. Parietal cells express high levels of Na—K-2Cl cotransporter on migrating into the gastric gland neck. Am J Physiol 1999; 276:G1273-G1278.
38. Geibel J P, Wagner C A, Caroppo R, Qureshi I, Gloeckner J, Manuelidis L, Kirchhoff P, Radebold K. The stomach divalent ion-sensing receptor scar is a modulator of gastric acid secretion. J Biol Chem 2001; 276:39549-39552.
39. Dufner M M, Kirchhoff P, Remy C, Hafner P, Muller M K, Cheng S X, Tang L Q, Hebert S C, Geibel J P, Wagner C A. The Calcium-Sensing Receptor (CaSR) acts as a modulator of gastric acid secretion in freshly isolated human gastric glands. Am J Physiol Gastrointest Liver Physiol 2005.
40. Schultheis P J, Clarke L L, Meneton P, Harline M, Boivin G P, Stemmermann G, Duffy J J, Doetschman T, Miller M L, Shull G E. Targeted disruption of the murine Na+/H+ exchanger isoform 2 gene causes reduced viability of gastric parietal cells and loss of net acid secretion. J Clin Invest 1998; 101:1243-1253.
41. Kirchhoff P, Andersson K, Socrates T, Sidani S M, Kosiek O, Geibel J P.
Characteristics of the K+-competitive H+,K+-ATPase Inhibitor AZD0865 in isolated rat gastric glands. Am J Physiol Gastrointest Liver Physiol 2006.
42. Andersson K, Carlsson E. Potassium-competitive acid blockade a new therapeutic strategy in acid-related diseases. Pharmacol Ther 2005; 108:294-307.
43. Dammann H G, Burkhardt F. Pantoprazole versus omeprazole: influence on meal-stimulated gastric acid secretion. Eur J Gastroenterol Hepatol 1999; 11:1277-1282.

### ADDITIONAL REFERENCES

1a. Sachs G. The parietal cell as a therapeutic target. Scand J Gastroenterol Suppl 1986; 118:1-10.
2a. Lorentzon P, Scott D, Hersey S, Wallmark B, Rabon E, Sachs G. The gastric H+,K+-ATPase. Prog Clin Biol Res 1988; 273:247-254.
3a. Wallmark B, Lorentzon P, Sachs G. The gastric H+,K(+)-ATPase. J Intern Med Suppl 1990; 732:3-8.
4a. Prinz C, Kajimura M, Scott D, Helander H, Shin J, Besancon M, Bamberg K, Hersey S, Sachs G. Acid secretion and the H,K ATPase of stomach. Yale J Biol Med 1992; 65:577-596.
5a. Scott D R, Helander H F, Hersey S J, Sachs G. The site of acid secretion in the mammalian parietal cell. Biochim Biophys Acta 1993; 1146:73-80.
6a. Sachs G, Prinz C, Loo D, Bamberg K, Besancon M, Shin J M. Gastric acid secretion: activation and inhibition. Yale J Biol Med 1994; 67:81-95.
7a. Hersey S J, Sachs G. Gastric-Acid Secretion. Physiological Reviews 1995; 75:155-189.
8a. Hirschowitz B I, Keeling D, Lewin M, Okabe S, Parsons M, Sewing K, Wallmark B, Sachs G. Pharmacological Aspects of Acid-Secretion. Digestive Diseases and Sciences 1995; 40:S3-S23.
9a. Wolfe M M, Welage L S, Sachs G. Proton pump inhibitors and gastric acid secretion. Am J Gastroenterol 2001; 96:3467-3468.
10a. Hersey S J, Sachs G. Gastric-Acid Secretion. Physiological Reviews 1995; 75:155-189.
11a. Hersey S J, Sachs G. Gastric-Acid Secretion. Physiological Reviews 1995; 75:155-189.
12a. Forte J G, Ly B, Rong Q, Ogihara S, Ramilo M, Agnew B, Yao X. State of actin in gastric parietal cells. Am J Physiol 1998; 274:C97-104.
13a. Urushidani T, Forte J G. Signal transduction and activation of acid secretion in the parietal cell. J Membr Biol 1997; 159:99-111.
14a. Geibel J P. Role of potassium in acid secretion. World J Gastroenterol 2005; 11:5259-5265.
15a. Blachar A, Federle M P. Gastrointestinal complications of laparoscopic roux-en-Y gastric bypass surgery in patients who are morbidly obese: findings on radiography and CT. AJR Am J Roentgenol 2002; 179:1437-1442.
16a. Peghini P L, Katz P O, Castell D O. Ranitidine controls nocturnal gastric acid breakthrough on omeprazole: a controlled study in normal subjects. Gastroenterology 1998; 115:1335-1339.
17a. Peghini P L, Katz P O, Bracy N A, Castell D O. Nocturnal recovery of gastric acid secretion with twice-daily dosing of proton pump inhibitors. Am J Gastroenterol 1998; 93:763-767.
18a. Sanders S W, Moore J G, Day G M, Tolman K G. Circadian differences in pharmacological blockade of meal-stimulated gastric acid secretion. Aliment Pharmacol Ther 1992; 6:187-193.
19a. Knauf F, Yang C L, Thomson R B, Mentone S A, Giebisch G, Aronson P S. Identification of a chloride-formate exchanger expressed on the brush border membrane of renal proximal tubule cells. Proc Natl Acad Sci USA 2001; 98:9425-9430.
20a. Dufner M M, Kirchhoff P, Remy C, Hafner P, Muller M K, Cheng S X, Tang L Q, Hebert S C, Geibel J P, Wagner C A. The calcium-sensing receptor acts as a modulator of gastric acid secretion in freshly isolated human gastric glands. Am J Physiol Gastrointest Liver Physiol 2005; 289:G1084-G1090.
21a. Kirchhoff P, Dave M H, Remy C, Kosiek O, Busque S M, Dufner M, Geibel J P, Verrey F, Wagner C A. An amino acid transporter involved in gastric acid secretion. Pflugers Arch 2006; 451:738-748.
22a. Waisbren S J, Geibel J, Boron W F, Modlin I M. Luminal perfusion of isolated gastric glands. Am J Physiol 1994; 266:C1013-C1027.
23a. Waisbren S J, Geibel J P, Modlin I M, Boron W F. Unusual permeability properties of gastric gland cells. Nature 1994; 368:332-335.
24a. Busque S M, Kerstetter J E, Geibel J P, Insogna K. L-type amino acids stimulate gastric acid secretion by activation of the calcium-sensing receptor in parietal cells. Am J Physiol Gastrointest Liver Physiol 2005; 289:G664-G669.
25a. Geibel J P, Wagner C A, Caroppo R, Qureshi I, Gloeckner J, Manuelidis L, Kirchhoff P, Radebold K. The stomach divalent ion-sensing receptor scar is a modulator of gastric acid secretion. J Biol Chem 2001; 276:39549-39552.
26a. Sachs G, Scott D, Reuben M. Omeprazole and the gastric mucosa. Digestion 1990; 47 Suppl 1:35-38.
27a. Sachs G, Wallmark B. The gastric H+,K+-ATPase: the site of action of omeprazole. Scand J Gastroenterol Suppl 1989; 166:3-11.
28a. Alino S F, Garcia D, Uvnas-Moberg K. On the interaction between intragastric pH and electrical vagal stimulation in causing gastric acid secretion and intraluminal release of gastrin and somatostatin in anesthetized rats. Acta Physiol Scand 1983; 117:491-495.
29a. Meulemans A L, Eelen J G, Schuurkes J A. NO mediates gastric relaxation after brief vagal stimulation in anesthetized dogs. Am J Physiol 1995; 269:G255-G261.
30a. Singh J. Prostaglandin release from rat stomach following vagal stimulation or administration of acetylcholine. Eur J Pharmacol 1980; 65:39-48.
31a. Aarimaa M, Soderstrom K O, Kalimo H, Inberg M, Nevalainen T. Morphology and function of the parietal cells after proximal selective vagotomy in duodenal ulcer patients. Scand J Gastroenterol 1984; 19:787-797.
32a. Amdrup E. The surgical treatment of duodenal ulcer. Schweiz Med Wochenschr 1979; 109:583-585.
33a. Waisbren S J, Modlin I M. The evolution of therapeutic vagotomy. Surg Gynecol Obstet 1990; 170:261-272.
34a. Abelo A, Eriksson U G, Karlsson M O, Larsson H, Gabrielsson J. A turnover model of irreversible inhibition of gastric acid secretion by omeprazole in the dog. J Pharmacol Exp Ther 2000; 295:662-669.
35a. Andersen J B, Andrade D V, Wang T. Effects of inhibition gastric acid secretion on arterial acid-base status during digestion in the toad Bufo marinus. Comp Biochem Physiol A Mol Integr Physiol 2003; 135:425-433.
36a. Gedda K, Scott D, Besancon M, Lorentzon P, Sachs G. Turnover of the gastric H+,K(+)-adenosine triphosphatase alpha subunit and its effect on inhibition of rat gastric acid secretion. Gastroenterology 1995; 109:1134-1141.
37a. Sachs G, Shin J M, Pratha V, Hogan D. Synthesis or rupture: duration of acid inhibition by proton pump inhibitors. Drugs Today (Barc) 2003; 39 Suppl A:11-14.
38a. Shamburek R D, Schubert M L. Pharmacology of gastric acid inhibition. Baillieres Clin Gastroenterol 1993; 7:23-54.
39a. Locke G R, III. Current medical management of gastroesophageal reflux disease. Thorac Surg Clin 2005; 15:369-375.
40a. Geibel J P. Secretion and absorption by colonic crypts. Annu Rev Physiol 2005; 67:471-490.

1. A method of increasing the pH of the gastric juices of the stomach of a patient or subject, said method comprising administering to said patient an effective amount of at least one zinc salt hydrate, preferably zinc sulfate heptahydrate alone or in combination with at least one additional zinc salt hydrate, and optionally, at least one additional pharmaceutically acceptable zinc salt (non-hydrate), said zinc salt hydrate being administered in a dose effective to produce a concentration within said gastric juices of about 0.3 μM to about 300 μM, preferably about 3 μM to about 30 μM.

2. The method according to claim 1 wherein said zinc salt hydrate is selected from the group consisting of zinc sulfate heptahydrate, zinc chloride monohydrate, zinc chloride dihydrate, zinc chloride trihydrate, zinc chloride quatrahydrate, zinc acetate dihydrate, zinc citrate dihydrate, zinc citrate trihydrate, zinc gluconate monohydrate, zinc gluconate dihydrate, zinc gluconate trihydrate, zinc lactate monohydrate, zinc lactate dihyrate, zinc lactate trihydrate, zinc bromide dihydrate, zinc fluoride dihydrate, zinc nitrate hydrate, zinc perchlorate hexahydrate, zinc picolinate dihydrate, zinc sulfate monohydrate, zinc tetrafluoroborate hydrate, zinc p-toluenesulfate hydrate, and mixtures thereof.

3. The method according to claim 1 wherein said zinc salt hydrate is selected from the group consisting of zinc sulfate heptahydrate, zinc chloride monohydrate, zinc chloride dehydrate, zinc chloride trihydrate, zinc chloride quatrahydrate, zinc acetate dihydrate, zinc gluconate monohydrate, zinc gluconate dihydrate, zinc gluconate trihydrate, zinc citrate dihydrate, zinc citrate trihydrate, zinc sulfate monohydrate and mixtures thereof.

4. The method according to claim 1 wherein said zinc salt hydrate is zinc sulfate heptahydrate which is used in the absence of any other zinc salt.

5. The method according to claim 1 wherein said additional zinc salt (non-hydrate) is selected from the group consisting of zinc acetate, zinc arginate, zinc butyrate, zinc chloride, zinc citrate, zinc formate, zinc fumarate, zinc gluconate, zinc glutarate, zinc glycerate, zinc glycolate, zinc histidinate, zinc lactate, zinc malate, zinc maleate, zinc picolinate, zinc propionate, zinc salicylate, zinc succinate, zinc sulfate, zinc tartrate, zinc undecylenate, zinc salt of 1,6 fluctose diphosphate and mixtures thereof.

6. The method according to claim 1 wherein said additional zinc salt is selected from the group consisting of zinc acetate, zinc ascorbate, zinc butryate, zinc carbonate, zinc citrate, zinc chloride, zinc iodide, zinc sulfate, zinc gluconate, zinc glycerate, zinc glycolate, zinc formate, zinc lactate, zinc malate, zinc maleate, zinc picolinate, zinc salicylate, zinc stearate, zinc succinate, zinc tartrate, zinc undecylenate, a zinc amino acid chelate and mixtures thereof.

7. The method according to claim 1 wherein said additional zinc salt is zinc chloride, zinc acetate, zinc gluconate, zinc lactate, zinc picolinate, zinc tartarate or mixtures thereof.

8. The method according to claim 1 wherein said zinc salt hydrate is coadministered with at least one proton pump inhibitor and/or an antacid.

9. The method according to claim 8 wherein said proton pump inhibitor is selected from the group consisting of omeprazole, esomeprazole, lansoprazole, pantoprazole and rabeprazole.

10. The method according to claim 1 wherein said pH of said gastric juices in said patient increases about 1 pH unit to about 2 pH units from the pH of gastric juice produced during secretagogue in a period of no greater than one hour.

11. The method according to claim 1 wherein said pH of said gastric juices in said patient increases at least about 1.5 pH units from the pH of gastric juice in said patient during secretagogue within a period no greater than about 30 minutes of administering said zinc sulfate heptahydrate.

12. The method according to claim 1 wherein said pH of said gastric juices in said patient increases to at least a pH within the range of 3.0-4.5 within a period no greater than about 20 minutes of administering said zinc salt(s).

13. A method of reducing the likelihood of or treating an ulcer developing in a patient at risk for an ulcer because of elevated acid release in the stomach of said patient comprising administering to said patient in need an effective amount of at least one zinc salt hydrate, preferably, zinc sulfate heptahydrate and optionally, at least one pharmaceutically acceptable zinc salt (non-hydrate) and optionally, at least one additional agent selected from the group consisting of a proton pump inhibitor, an antacid, an H2 blocker, an anti-H. pylori agent, acetyl salicyclic acid, a blood thinning agent, copper and mixtures thereof.

14. The method according to claim 13 wherein said zinc salt hydrate is selected from the group consisting of zinc sulfate heptahydrate, zinc chloride monohydrate, zinc chloride dehydrate, zinc chloride trihydrate, zinc chloride quatrahydrate, zinc acetate dihydrate, zinc citrate dihydrate, zinc citrate trihydrate, zinc gluconate monohydrate, zinc gluconate dihydrate, zinc gluconate trihydrate, zinc lactate monohydrate, zinc lactate dehydrate, zinc lactate trihydrate, zinc picolinate dihydrate, zinc bromide dihydrate, zinc fluoride dihydrate, zinc nitrate hydrate, zinc perchlorate hexahydrate, zinc sulfate monohydrate, zinc tetrafluoroborate hydrate, zinc p-toluenesulfate hydrate, and mixtures thereof.

15. The method according to claim 13 wherein said zinc salt hydrate is selected from the group consisting of zinc sulfate heptahydrate, zinc chloride monohydrate, zinc chloride dehydrate, zinc chloride trihydrate, zinc chloride quatrahydrate, zinc acetate dihydrate, zinc gluconate monohydrate, zinc gluconate dihydrate, zinc gluconate trihydrate, zinc citrate dehydrate, zinc citrate trihydrate, zinc lactate monohydrate, zinc lactate dehydrate, zinc lactate trihydrate, zinc picolinate dihydrate, zinc sulfate monohydrate and mixtures thereof.

16. The method according to claim 13 wherein said zinc salt hydrate is zinc sulfate heptahydrate which is used in the absence of any other zinc salt.

17. The method according to claim 13 wherein a mixture of zinc sulfate heptahydrate and at least one additional zinc salt (non-hydrate) is administered to said patient.

18. The method according to claim 17 wherein said additional zinc salt is selected from the group consisting of zinc acetate, zinc ascorbate, zinc butryate, zinc carbonate, zinc citrate, zinc chloride, zinc iodide, zinc sulfate, zinc gluconate, zinc glycerate, zinc glycolate, zinc formate, zinc lactate, zinc malate, zinc maleate, zinc picolinate, zinc salicylate, zinc stearate, zinc succinate, zinc tartrate, zinc undecylenate, a zinc amino acid chelate and mixtures thereof.

19. (canceled)

20. The method according to claim 13 wherein said zinc salt(s) is combined with an effective amount of at least one proton pump inhibitor.

21. The method according to claim 20 wherein said proton pump inhibitor is selected from the group consisting of omeprazole, esomeprazole, lansoprazole, pantoprazole and rabeprazole and mixtures thereof.

22. A method of treating a patient for a disease state or condition selected from the group consisting of gastroesophageal reflux disease, (GERD), non-erosive reflux disease (NERD), Zollinger-Ellison syndrome (ZE syndrome), ulcer disease and gastric cancer comprising administering to said patient an effective amount of an effective amount of at least one zinc salt hydrate, preferably, zinc sulfate heptahydrate and optionally, at least one pharmaceutically acceptable zinc salt (non-hydrate).

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A method of inhibiting vacuolar H⁺-ATPase, H⁺, K⁺-ATPase or both H⁺-ATPase and H⁺, K⁺-ATPase in the stomach of a patient resulting in an increase in the pH of gastric juices of said patient comprising administering to said patient an effective amount of at least one zinc salt hydrate, preferably zinc sulfate heptahydrate, alone or in combination with another zinc salt hydrate and optionally, at least one additional pharmaceutically acceptable zinc salt.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A pharmaceutical composition comprising an effective amount of a zinc salt hydrate, preferably zinc sulfate heptahydate, alone or in combination with at least one additional zinc salt hydrate and optionally at least one additional pharmaceutically acceptable zinc salt non-hydrate in combination with an effective amount of a proton pump inhibitor, an antacid, an H2 blocker, an anti-H. pylori agent, a cytoprotective agent, acetyl salicylic acid, an antibiotic, a blood thinning agent or mixtures thereof, optionally in combination with a pharmaceutically acceptable carrier, additive or excipient.

52. (canceled)

53. (canceled)

54. (canceled)

55. (canceled)

56. A method of treating an H. pylori infection in a patient comprising administering to said patient an effective amount of at least one zinc salt hydrate, preferably zinc sulfate heptahydrate alone or in combination with at least one addition zinc salt hydrate and optionally, at least one additional pharmaceutically acceptable zinc salt non-hydrate, further optionally in combination with an anti-H. pylori agent.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. A pharmaceutical composition comprising an effective amounts of at least one zinc salt hydrate, preferably, zinc sulfate heptahydrate, alone or in combination with an additional zinc salt hydrate, and optionally, at least one additional pharmaceutically acceptable water soluble zinc salt in combination with at least one therapeutic agent which is favorably orally administered in combination with said zinc salt hydrate, preferably zinc sulfate heptahydrate and optional zinc salt, optionally in combination with a pharmaceutically acceptable carrier, additive or excipient.

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119.-130. (canceled)
